SEQUENCE LISTING (72 sequences)

SEQ ID NO: 1 - DNA, 279 bp, Issatchenkia orientalis
  misc_feature (1)..(279): ARS-1
ctttggttctgataggaataaaaaagcagataaacaatttgaaaaagcaaaaaagaactgagcccgatgcggggctcgaacccgcagccttttgattgcacttccttaacagaagaaatcttaagagtcaaacgctctaccgattgagctaaccaggcttttcttgaaattaaacttttatggatttatatgttgtttgtagatatctgatgcctgctcactatttaatcattatatgaaacaaaagaacatctttcttcttaaccattgtataaatat

SEQ ID NO: 2 - DNA, 704 bp, Issatchenkia orientalis
  misc_feature (1)..(704): ARS-2
atttatgaaagcggaagttgagaagtggcagaacaattttgaaatggtagctgattctaatgatgttctactgagaagtattaacttggttgatcgtccattgctagatgattatttcagagtcaccactacaactaaatcgctgtttttaagaacgttgcctatttcaatatttgatattgcagtagagaaagaaaacattgtaattatgtaatgtgaatctttaagagttgtcactgagtcactgaataacagctaaacttaattcttcaatacaaaaaatagaacagcttcaagaaaaataatggacgagaccggagtcgaaccgatgacctttcgcttgcaaggcgaacgcgctaccaactacgccacacgcccagtaaaataagaatggtccctagcaggatcgaactgctgatcttggcgttattagcaccacgccttaaccaactgggccaagggaccttttcttgaagaggaagttttacggttttaaaccactaacacaaaaaatatgtcttagtttgtatttattctcttatttttttttgtattagtaaattaatgagaaatgaaaatgaacaatgtgaataattgtctggaaaataatcttttcacgtagtgataaaaaattcaaagtttcaagagaaacgaatatatcaaaatgtcgaaataaaaattgttcaacttgacagattaaaact

SEQ ID NO: 3 - skipped

SEQ ID NO: 4 - DNA, 90 bp, Issatchenkia orientalis
cgaacccgcagccttttgattgcacttccttaacagaagaaatcttaagagtcaaacgctctaccgattgagctaaccaggcttttcttg

SEQ ID NO: 5 - DNA, 45 bp, Issatchenkia orientalis
taagagtcaaacgctctaccgattgagctaaccaggcttttcttg

SEQ ID NO: 6 - DNA, 37 bp, Artificial Sequence
  Consensus sequence for ARS-1
  misc_feature (7)..(7): n is a, c, g, or t
  misc_feature (9)..(10): n is a, c, g, or t
  misc_feature (12)..(12): n is a, c, g, or t
  misc_feature (31)..(31): n is a, c, g, or t
  misc_feature (34)..(34): n is a, c, g, or t
taagagncnnangctctaccgattgagctanccnggc

SEQ ID NO: 7 - DNA, 37 bp, Artificial Sequence; identical to SEQ ID NO: 6
  Consensus sequence for ARS-1
  misc_feature (7)..(7): n is C or T
  misc_feature (9)..(9): n is A or T
  misc_feature (10)..(10): n is A or C
  misc_feature (12)..(12): n is C or T
  misc_feature (31)..(31): n is G or A
  misc_feature (34)..(34): n is G or A

SEQ ID NO: 8 - DNA, 18 bp, Artificial Sequence
  Highly conserved sequence
gctctaccgattgagcta

SEQ ID NO: 9 - DNA, 43 bp, Candida ethanolica
ttaagagtcaaacgctctaccgattgagctagccaggcttttc

SEQ ID NO: 10 - DNA, 41 bp, Candida intermedia
ttaagagtcaaatgctctaccgattgagctaaccaggcttt

SEQ ID NO: 11 - DNA, 46 bp, Candida sorboxylosa
ttaagagtcaaacgctctaccgattgagctaaccaggcttttgttg

SEQ ID NO: 12 - DNA, 47 bp, Candida tanzawaensis
cttaagagtcaaacgctctaccgattgagctaaccaggcctttcttg

SEQ ID NO: 13 - DNA, 40 bp, Debaryomyces hansenii
ttaagagtcaaacgctctaccgattgagctagccaggctt

SEQ ID NO: 14 - DNA, 40 bp, Leptosphaeria biglobosa
taagagtctcacgctctaccgattgagctaaccaggcttt

SEQ ID NO: 15 - DNA, 38 bp, Leptosphaeria maculans
taagagtctcacgctctaccgattgagctaaccaggct

SEQ ID NO: 16 - DNA, 41 bp, Metschnikowia australis; identical to SEQ ID NO: 10

SEQ ID NO: 17 - DNA, 47 bp, Millerozyma farinosa
ttaagagtcaaacgctctaccgattgagctagccaggcttcttgttg

SEQ ID NO: 18 - DNA, 46 bp, Nakazawaea peltata
taagagtcaaacgctctaccgattgagctagccaggctatttcttg

SEQ ID NO: 19 - DNA, 47 bp, Pichia kudriavzevii
cttaagagtcaaacgctctaccgattgagctaaccaggcttttcttg

SEQ ID NO: 20 - DNA, 46 bp, Pichia membranifaciens; identical to SEQ ID NO: 11

SEQ ID NO: 21 - DNA, 47 bp, Pichia sorbitophila
ttaagagtcaaacgctctaccgattgagctagccaggcttgttcttg

SEQ ID NO: 22 - DNA, 48 bp, Scheffersomyces lignosus
cttaagagtcaaacgctctaccgattgagctaaccaggcatgtttctt

SEQ ID NO: 23 - DNA, 46 bp, Scheffersomyces shehatae
ttaagagtcaaacgctctaccgattgagctaaccaggcatttgttg

SEQ ID NO: 24 - DNA, 45 bp, Scheffersomyces stipitis
taagagtcaaacgctctaccgattgagctaaccaggcatttcttg

SEQ ID NO: 25 - DNA, 40 bp, Spathaspora girioi
cttaagagtcaaacgctctaccgattgagctaaccaggct

SEQ ID NO: 26 - DNA, 45 bp, Spathaspora gorwiae
cttaagagtcaaacgctctaccgattgagctaaccaggcattttc

SEQ ID NO: 27 - DNA, 41 bp, Spathaspora hagerdaliae
taagagtcaaacgctctaccgattgagctaaccaggctttt

SEQ ID NO: 28 - DNA, 42 bp, Spathaspora passalidarum
ttaagagtcaaacgctctaccgattgagctaaccaggctttt

SEQ ID NO: 29 - DNA, 44 bp, Sugiyamaella xylanicola
taagagtcaaacgctctaccgattgagctaaccaggcttttctt

SEQ ID NO: 30 - DNA, 46 bp, Wickerhamia fluorescens; identical to SEQ ID NO: 11

SEQ ID NO: 31 - DNA, 36 bp, Artificial Sequence
  Consensus sequence from alignment of SEQ ID NOs: 9-30
  misc_feature (8)..(9): n is a, c, g, or t
  misc_feature (11)..(11): n is a, c, g, or t
  misc_feature (30)..(30): n is a, c, g, or t
taagagtnnangctctaccgattgagctanccaggc

SEQ ID NO: 32 - DNA, 36 bp, Artificial Sequence; identical to SEQ ID NO: 31
  Consensus sequence from alignment of SEQ ID NOs: 9-30
  misc_feature (8)..(8): n is A or T
  misc_feature (9)..(9): n is A or C
  misc_feature (11)..(11): n is C or T
  misc_feature (30)..(30): n is A or G

SEQ ID NO: 33 - DNA, 858 bp, Issatchenkia orientalis
  misc_feature (1)..(858): Comprises TEF1 promoter
ctgcaactacagagccattccttcacatgtatagaatatacacataaagaagagatgacgattttacatcaagaaaaaaacagccatttctgcaaacttttaggtttagccacagtttttcaacagaaaaaaagaagagttatgtttttattttttttaccttcgaaagctatactataaaagctactttccacatttttcagatgaatagcaaccccagttacgtagatgtgttttgggtcacctgcatagaaggtatttgaaacatcatgaaaactgtttcaccctctgtgaagcataaacactagaaagccaatgaagagctctacaagcctcatatgggttcaatgggtctgcaatgaccgcatacgggcttggacaattaccttctattgaatttctgagaagagatacatctgaccagcaatgtaagcagacaatcccaattctgtaaacaacctctttgtccataattccccatcagaagagtgaaaaatgccctcaaaatgcatgcgccacacccacctctcaactgcactgcgccacatctgagggtcctttcaggggtcgactaccccggacacctcgcagaggagcgacgtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttctttacgttttcttttcaacttttttttactttttccttcaacagacaaatctaacttatatatcaca

SEQ ID NO: 34 - DNA, 861 bp, Issatchenkia orientalis
  misc_feature (1)..(861): Comprises TDH3 Promoter
gttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtgtgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaa

SEQ ID NO: 35 - DNA, 845 bp, Issatchenkia orientalis
  misc_feature (1)..(845): Comprises PGK1 Promoter
ccaagaatgccggtgatatgatcaacaaatactccatcttatacaacagaaccagacaagccgtcattactaacgaattggttgatattattactggtgcttcctccttgaactagatgtgctgaatgccatggcaactcagagtcctacattgggactctgaaattctctaggagcatgcacacgtaatatacgtgtctgtctccacgccgatatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgccaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggagtgggggggggggggggaaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaaca

SEQ ID NO: 36 - DNA, 829 bp, Issatchenkia orientalis
  misc_feature (1)..(829): Comprises PGI1 Promoter
cctacacagacattactagccgtcattagacgtagacttatacacctctcgatgggttctcatggcgccttttgtcaacctgtgtctctttccaggacagcgtcggtagcatccacaatgtccatctcagacaccgtcgatcaccatcaactgccgccactacaatttccatcacagacacagctaatacaaccaatccaaccgccaccgccaccacgcccacaactctctctggcgaatccaagggcccttcgctcgccatctatatcttcaacgaacaacggaattacaaacatgggcagtagttcaaacaatctccagactctcaactctctctcgctatcgttgaaacatccacagttccaaggcctattctccccactggatgtccacagtccgtacgaacagaacgttccttccccactggcccccaccgttccggctgttccgggaaccgcaccttcattcgagtcggacgatctctacaatgcaacggctgcccgcaaaagagactctctcaagatgaagagaagatagacgctacatcattgtctgtgcagtacctaatatatagtacttggtataaggtataataaaactataaaattataataatcttaataataataaccatattaatggaaggatgaggcccgatgtccttttttttgcctttctactatagtgcttacattgtgtataaattctcatgattttccagctcgcatgtttttcttataaaaatagcccagaccccatatctgcccataactttatattccgttcccggaccaacttgagtaatacactcactacaaatagcgccgtc

SEQ ID NO: 37 - DNA, 863 bp, Issatchenkia orientalis
  misc_feature (1)..(863): Comprises PFK1 Promoter
ggcagtcctaaagcaaggcattgatcgatcaaaattcaccttcaaggcatggggccaaccttatttgtgtgcaatcactctaacggttgttttcatcatgatatggatcgatggttattatgtcttcttgccaggtagctggtcgacatcgaccttcttgtttacatatttgatgtgttttgttgactttggtattgtcgttgcgtggaaattcattaaaggcacaaaatatagatccaacccaatggaagtggacttacatactggtttggaggaagttattttacatgagttgatgcttgagaagaagagggcattgtatcaacagaaaagtccaatggctagaacatgggaaagattgaatgaactcttattcggcaagaattaagctcctcccttaaagttctactttttccttctattttataccaccctactccttcaattactattatttttcgctgttttgctttatttgatctacaactgtataactgacaagacctgtaataaatgcggctttaatcatttaatttcttttccgaggatcacgtgcaagtcccagagatggactactaaccgtgtgtgagactggtacaaaaaagacgaaatggaatacattatagagatgatgaggatttgcccaaagtggtgggaaaaaaaaaacatgggctgacgaaggaaagctgcactccgaagagcaccgcagtgcgtgagtttgttgcggacgcaatatatatatatataatctacctctggtgcatattttcttctttcttatagtgcccggacagtttcagtttgttgcttcggttataaaagaagaaacaaaaaaaagcggttagcaacaacagttcgcaatc

SEQ ID NO: 38 - DNA, 846 bp, Issatchenkia orientalis
  misc_feature (1)..(846): Comprises PDC1 Promoter
cagctctgtgttgaagagacgtacaagatccgcctttctgtcccttggcgacgccctgatcccatggcttgccaacagcgggcgaagatccttcacccgcagcgtccatggattgaactcgggcgataggtactgtgacgagtccatcggttcctgtcagatgggatactcttgacgtggaaaattcaaacagaaaaaaacccccaataatgaaaaataacactacgttatatccgtggtatcctctatcgtatcgtatcgtatcgtagcgtatcgtaccgtaccgtattacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgatgcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaat

SEQ ID NO: 39 - DNA, 850 bp, Issatchenkia orientalis
  misc_feature (1)..(850): Comprises HHF1 Promoter
ggttctagccatggtgtggttatattttatttggagtatgtaattaataaaggaacacacaagggtaaaataagcaaaaaataaaataaaataaaataaacttggaaaaaataaagggcaaaaaaaagtccaataaaaaagagcggggggttcacggtatttaaataattttccaagcggaaggtggaacaatagcagatggccaataaaaaaaaagaaacaccacagctgatggaaatggcctgttagtacaaagggggggaaacggcagtctcgtagcagccaaaaaaaagggcgcgaattctcgtgcttacgttccccattgaattaagagtttcttaactatgtcgaatgcgcgtgctgaaaactacggctaaacaagtgtagttgacatcacgcaaggtttgcccctctcctaatttcggtgaggttttacgcgataaaaaaaaattgtagagatggagaaaaaatttctttaaactgtcaatggaaacaggaggaaaagaaactgttgaaagacaaaaatcgaggtgtgcggtttgagagctgcgcctatgcgggcgcaagttcgggaacggtgtaaggaagggaagggggcgtttcagataggtacatttgtatgtcattgttggtattgtatacatttctcgccatcgtaccccattgtaaatggatgagtctctccatgtaaatgtaaaatttgatgagtgagaatatacgacaattcctttaaaaggggccaatgtccgcactggatttagggttgatataagtcacttttaataggagaatttttccttattgtccttgtattgtacacaacaaaagaaacccatacaacagatataca

SEQ ID NO: 40 - DNA, 881 bp, Issatchenkia orientalis
  misc_feature (1)..(881): Comprises ENO1 Promoter
ggagaaatgcagacagtcaatgaacacaactgtctcaatatgcatctatgcacatgcacacacgcacacatcacaggtacccctacaaagagaggtcccttgataatgtttcattaccacgtggcatccccccccccaataaacaagtggccgagttcccctgttgcagaggaggacaaaagaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgagggggggggggggtggtatataaagacaggagatgtccaccccaagagagaggaagaagttggaactttagaagagagagataactttccccagtgtccatcaatacacaaccaaacacaaactctatatatacacatataaccccctccaaccaaaca

SEQ ID NO: 41 - DNA, 832 bp, Issatchenkia orientalis
  misc_feature (1)..(832): Comprises CCW12 Promoter
ctcttctgacgggtttcctgtcgcttgcgagtgcgtcagctgcatttgtcaagagatacgataactcaacatcttcgtgtgtgccaacaacgatctcttctacttcgattgtttctgtttacggtcctcagtttactggtgtgccatcaaataagtacgttgttccaaccgttgttggtgacttggtttcaaacgatgctccagaattcaccgtttacattcctcagtccttgtacaatttaaccggtttggaattagagttaacttcagcggttggtttcgatgtgtcaccagaatcttatactttatattcaggtgacattggcgattttgtttacccaggccaaatttatgaaacatcaaatactggtttggtttttgacggtagaacgcaagatcctttactaaagattaccattattggtatccctgaagcaaaccaaccagttttcattgctgattttgttctcactttagatgtaatttcatcatctggcttacaaaagagggacaccttaacttttaatttgtcattctctattaagaattcggcatatatctcctcgtcttctgttgcatcaacaagtgaatcctcccaagctactactggtgctactactggtgctactactggtgctactactggtgaaactactggtgctactactggtgctactactggtgcttactggtgctactactggtgctactactggtgctactactggtgaaactaccggagctactaccggagctactggtgaaactaccggagctactaccggagctactaccggtgctactactggtgcaattacttctgagctcacta

SEQ ID NO: 42 - DNA, 894 bp, Issatchenkia orientalis
  misc_feature (1)..(894): Comprises ACT1 Promoter
gtggattatcgttattgaggtggtcatttatcattgacgactttctcaagaagcggcactttttcaccttcaccattccccccctttccgttccttgttaactaacaacttctttccttgtctcctctctatttgataccttggtctccctgtgtttgtttttgtttgtggtggttgtgtgcacacgtgtatgtttgtttgtttgtgtgtgtgttttgttttatcttttcgtgattttgagacaattgactcttcagttaaagacgggaatttatggattcattgatagtttatttgatgatatatggtctgtcatatatatttgaaggaaaatttatgaagaacctcttttagacacaaaccatcaactagtttaatctggatacaacttatcaagatattccttttatttggtttgattgatttgatgaagatccagatatacattgtctttgtttttaccaaagtaatcaagggttctcaattccttagtggttatatttatagcttgtgattatgtttttccccccccgtgtttgccaatctcacaactatacaaaaagaaatagagaaatatgacaattatgccataatacaaaaagtggtattatccttattttttattccatccaccaattccttctattcagtagaaacaaaaagcattttctaacttatagttcattttgaccaattcacgttcttggtgattttgtttatttgtccattacgaagagaatcacaaaacagaacaaacagtacaaacaaacaaacaaacaaacaaacaaacaaaggagaaaacattttggaaaataaaattaaagcaacgtactaacatttaaccattccccctattattgttttctatagttagttattgataatggttctggt

SEQ ID NO: 43 - DNA, 217 bp, Issatchenkia orientalis
  misc_feature (1)..(217): Comprises ADH1 Terminator
tgggctgacttgggtgtactggtgtgacgtttttatgtgtatattgatatgcatgggggatgtatagtgatgaggagtagagtatataacgaaatgaaatgaaataatatgataagataagataagataagatcaaataagataatataagatgcgacatgaggagttcaatgtagcatactacacgatgctgcagtacaactctgatacgctagac

SEQ ID NO: 44 - DNA, 209 bp, Issatchenkia orientalis
  misc_feature (1)..(209): TDH3 Terminator
gcggcgaatctctggctcatgggggatatcctctttggcttttttttcccattctctttgttttgattatctaatgactcattgggaggattttctcacttcaagcttttttttcttgcactctttcataactccagctctctctaactgaggctacaatgccttttaacgaacttatgagacgtttctaaattatataggtatatgcc

SEQ ID NO: 45 - DNA, 73 bp, Issatchenkia orientalis
  misc_feature: tRNA Threonine
gctcgtatggccaagttggtaaggcgctacactagtaatgtagcgatcctcagttcgactctgagtgcgagca

SEQ ID NO: 46 - DNA, 84 bp, Issatchenkia orientalis
  misc_feature: tRNA Leucine
ggagggatggccgagtggtctaaggcggcagacttaagatctgttggacgcatgtccgcgcgagttcgaacctcgcttccttca

SEQ ID NO: 47 - DNA, 72 bp, Issatchenkia orientalis
  misc_feature: tRNA Proline
gggttaatggtctagtggtatgattctcgctttgggtgcgagaggccctgggttcaattcccagttgacccc

SEQ ID NO: 48 - DNA, 74 bp, Issatchenkia orientalis
  misc_feature: tRNA Methionine
gctttggtggcccagttggttaaggcgtcagtctcataatctgaagatcgcgagttcgaatctcgcctagagca

SEQ ID NO: 49 - DNA, 72 bp, Issatchenkia orientalis
  misc_feature: tRNA Glutamine
tccgatatagtgtaacggctatcacggtccgctttcaccgggcagacccgggttcgactcccggtatcggaa

SEQ ID NO: 50 - DNA, 72 bp, Issatchenkia orientalis
  misc_feature: tRNA Glutamate
aggtcgtacccggattcgaaccggggttggtcggatcaaaaccgacagtgataaccactacactatacaacc

SEQ ID NO: 51 - DNA, 74 bp, Issatchenkia orientalis
  misc_feature: tRNA Valine
ggtcggatggtctagttggttatggcatatgcttaacacgcataacgtccccagttcgatcctgggttcgatca

SEQ ID NO: 52 - DNA, 82 bp, Issatchenkia orientalis
  misc_feature: tRNA Serine
ggcaatttgtccgagtggttaaggagaaagattagaaatcttttgggctttgcccgcgcaggttcgaatcctgcagttgtcg

SEQ ID NO: 53 - DNA, 72 bp, Issatchenkia orientalis
  misc_feature: tRNA Histidine
gccgttctagtatagtggtcagtacgcatcgttgtggccgatgagacccaggttcgattcctgggaacggca

SEQ ID NO: 54 - DNA, 72 bp, Issatchenkia orientalis
  misc_feature: tRNA Phenylalanine
gcgggcttagctcagtgggagagcgccagactgaagatctggaggccctgtgttcgatccacagagctcgca

SEQ ID NO: 55 - DNA, 73 bp, Issatchenkia orientalis
  misc_feature: tRNA Arginine
gcccgtgtagcgtaatggttaacgcgtttgacttctaatcaaaagattctgggttcgactcccagcatgggtg

SEQ ID NO: 56 - DNA, 73 bp, Issatchenkia orientalis
  misc_feature: tRNA Alanine
gggcgtgtggcgtagttggtagcgcgttcgccttgcaagcgaaaggtcatcggttcgactccggtctcgtcca

SEQ ID NO: 57 - DNA, 74 bp, Issatchenkia orientalis
  misc_feature: tRNA Isoleucine
ggtcccttggcccagttggttaaggcgtggtgctaataacgccaagatcagcagttcgatcctgctagggacca

SEQ ID NO: 58 - DNA, 72 bp, Issatchenkia orientalis
  misc_feature: tRNA Asparagine
ctccgagaccgggaattgaacccgggtctcccgcgtgacaagcggaaattctagccactaaactatctcgga

SEQ ID NO: 59 - DNA, 72 bp, Issatchenkia orientalis
  misc_feature: tRNA Cysteine
agcccgcggccgggtttgaaccggcgaccaacagatttgcaatctgctgctctaccactgagctacgcgtgc

SEQ ID NO: 60 - DNA, 72 bp, Issatchenkia orientalis
  misc_feature: tRNA Tryptophan
ggggctatggctcaatggtagagctttcgactccagatcgaagggttgcaggttcgattcctgttggcctca

SEQ ID NO: 61 - DNA, 176 bp, Issatchenkia orientalis
  misc_feature: tRNA Threonine (SEQ ID NO: 45) + 100-bp 5′ genomic DNA sequence
gatatccttatgctttgcaaatcacatatgcagtagtagaatagacaaactggaccacaattgcattttatagaatagtggaattttggacacacaacaaaccgctcgtatggccaagttggtaaggcgctacactagtaatgtagcgatcctcagttcgactctgagtgcgagca

SEQ ID NO: 62 - DNA, 180 bp, Issatchenkia orientalis
  misc_feature: tRNA Leucine (SEQ ID NO: 46) + 100-bp 5′ genomic DNA sequence
ctcagggaattaacgtataaaaatatataagatagaaataagcaagaatcagattcaagagtaggtactaccagccccctatgaattttatcaattggagggatggccgagtggtctaaggcggcagacttaagatctgttggacgcatgtccgcgcgagttcgaacctcgcttccttca

SEQ ID NO: 63 - DNA, 199 bp, Issatchenkia orientalis
  misc_feature: tRNA Proline (SEQ ID NO: 47) + 100-bp 5′ genomic DNA sequence
atgttctcagagaattaacgtataaaaatatataagatataaataagcaataatcagattctaaagtacgcaccaccagcaacactttcacattcataagcccagcaacactttcacattcataagcgggttaatggtctagtggtatgattctcgctttgggtgcgagaggccctgggttcaattcccagttgacccc

SEQ ID NO: 64 - DNA, 89 bp, Saccharomyces cerevisiae
  misc_feature: S. cerevisiae tRNA Tyrosine
ctctcggtagccaagttggtttaaggcgcaagactgtaatttatcactacgaaatcttgagatcgggcgttcgactcgcccccgggaga

SEQ ID NO: 65 - DNA, 89 bp, Saccharomyces cerevisiae
  misc_feature: S. cerevisiae tRNA Phenylalanine
ggatttagctcagttgggagagcgccagactgaagaaaaacttcggtcaagtcatctggaggtcctgtgttcgatccacagaattcgca

SEQ ID NO: 66 - DNA, 9 bp, Artificial Sequence
  I. orientalis tRNA consensus sequence TGGnCnAGT
  misc_feature (4)..(4): n is a, c, g, or t
  misc_feature (6)..(6): n is a, c, g, or t
tggncnagt

SEQ ID NO: 67 - DNA, 9 bp, Artificial Sequence
  I. orientalis tRNA consensus sequence GTTCnAnnC
  misc_feature (5)..(5): n is a, c, g, or t
  misc_feature (7)..(8): n is a, c, g, or t
gttcnannc

SEQ ID NO: 68 - DNA, 9 bp, Artificial Sequence
  I. orientalis tRNA consensus sequence GnTCnAnnC
  misc_feature (2)..(2): n is a, c, g, or t
  misc_feature (5)..(5): n is a, c, g, or t
  misc_feature (7)..(8): n is a, c, g, or t
gntcnannc

SEQ ID NO: 69 - DNA, 9 bp, Artificial Sequence
  I. orientalis tRNA consensus sequence GTTCnAnnC
  misc_feature (5)..(5): n is a, c, g, or t
  misc_feature (7)..(8): n is a, c, g, or t
gttcnannc

SEQ ID NO: 70 - DNA, 73 bp, Artificial Sequence
  Consensus sequence for ARS-2
  misc_feature (9)..(12): n is a, c, g, or t
  misc_feature (15)..(15): n is a, c, g, or t
  misc_feature (22)..(23): n is a, c, g, or t
  misc_feature (25)..(25): n is a, c, g, or t
  misc_feature (28)..(28): n is a, c, g, or t
  misc_feature (32)..(32): n is a, c, g, or t
  misc_feature (34)..(37): n is a, c, g, or t
  misc_feature (40)..(40): n is a, c, g, or t
  misc_feature (42)..(42): n is a, c, g, or t
  misc_feature (44)..(45): n is a, c, g, or t
tggacgagnnnngantcgaacnnangancttncnnnngcnangnnaacgcgctaccaactacgccacacgccc

SEQ ID NO: 71 - DNA, 73 bp, Artificial Sequence
  Consensus sequence for ARS-2
  misc_feature (28)..(28): n is a, c, g, or t
  misc_feature (45)..(45): n is a, c, g, or t
tggacgagaccggagtcgaaccgatganctttcgcttgcaaggcnaacgcgctaccaactacgccacacgccc

SEQ ID NO: 72 - DNA, 28 bp, Artificial Sequence
  Consensus sequence for ARS-2
aacgcgctaccaactacgccacacgccc

(19) United States
(12) Patent Application Publication
(10) Pub. No.: US 2020/0277614 A1
(43) Pub. Date: Sep. 3, 2020

(54) TOOLS AND METHODS FOR GENOME EDITING ISSATCHENKIA ORIENTALIS AND OTHER INDUSTRIALLY USEFUL YEAST

(71) Applicant: LCY BIOSCIENCES INC., Samia, Ontario (CA)

(72) Inventor: Owen RYAN, Minneapolis, MN (US)

(21) Appl. No.: 16/612,288

(22) PCT Filed: May 14, 2018
(86) PCT No.: PCT/CA2018/050569

§ 371 Date: Nov. 8, 2019

Related U.S. Application Data
(60) Provisional application No. 62/505,451, filed on May 12, 2017.

(51) Int. Cl. C12N 15/81

(52) U.S. Cl. CPC C12N 15/815; C12N 2830/36; C12N 2820/55; C12N 2800/24

(57) ABSTRACT

The present description relates to genetic tools and methods to facilitate transformation and genetic engineering of industrially-useful yeast/fungal species, such as Issatchenkia orientalis, for which a robust set of genetic tools such as stably inherited and maintained plasmids and functional control sequences is presently lacking. Thus, the present description relates to autonomously replicating sequences (ARSs), RNA polymerase II/III promoters, RNA polymerase II/III terminators, expression cassettes, and vectors comprising same are described herein, as well as uses and methods relating to same, which are functional in I. orientalis.

The present description relates to autonomously replicating sequences (ARSs), promoters, terminators, and vectors that facilitate transformation and/or genome editing in yeast/fungal extremophiles, such as Issatchenkia orientalis, as well as methods and uses relating thereto.

The present description refers to a number of documents, the contents of which are herein incorporated by reference in their entirety.

### BACKGROUND

Yeast extremophiles have been exploited to function as powerful industrial microbes and biocatalysts because of their high tolerance to process conditions (e.g., low pH). Issatchenkia orientalis is an example of a naturally occurring acidophilic Ascomycete yeast which has been used for industrial applications, such as for the bioproduction of organic acids. Unlike model organisms such as Saccharomyces cerevisiae, significant barriers to perform genetic and genomic engineering in these extremophiles exist, as there is a lack of robust genetic tools such as stably inherited and maintained plasmids. In fact, many of the genetic tools developed and optimized for model organisms like S. cerevisiae simply do not function in many industrially useful yeast/fungal extremophiles, rendering the engineering of these organisms as difficult, laborious, and time-intensive processes. Thus, there is a need for novel genetic tools and methods to facilitate the genomic engineering of industrially useful extremophiles such as I. orientalis.

### SUMMARY

The present description relates to genetic tools and methods to facilitate transformation and/or genome editing in industrially-useful yeast/fungal species, such as Issatchenkia orientalis. More specifically, autonomously replicating sequences (ARSs), RNA polymerase II and III promoters, RNA polymerase II and III terminators, expression cassettes, and vectors comprising same are described herein, as well as uses and methods relating thereto.

In some aspects, the present description relates to a recombinant DNA molecule for expressing a non-polypeptide-encoding RNA (ncRNA) in host yeast or fungal cells, the recombinant DNA molecule comprising an expression cassette comprising: (i) an RNA polymerase III promoter sequence comprising a tRNA sequence from Issatchenkia orientalis (Pichia kudriavzevii or Candida krusei), or a variant or fragment of said tRNA sequence having RNA polymerase III promoter activity in I. orientalis cells; (ii) an ncRNA polynucleotide sequence encoding the ncRNA to be expressed in the host yeast or fungal cells; and (iii) an RNA polymerase III terminator sequence, wherein the RNA polymerase III promoter and terminator sequences enable transcription of said ncRNA polynucleotide when introduced into the host yeast or fungal cells, and wherein the expression cassette is non-native, exogenous, or heterologous with respect to the host yeast or fungal cells, and/or the ncRNA polynucleotide is heterologous with respect to the RNA polymerase III promoter and/or RNA polymerase III terminator. In embodiments, the tRNA sequence, or the variant or fragment thereof, may comprise the consensus sequence of SEQ ID NO: 66, 67, 68 or 69, and/or may be or may comprise a sequence at least 60%, 65%, 70%, 75%, 80%, 85%, 90%, 95%, or 99% identical to any one of SEQ ID NOs: 45-63. In embodiments, the RNA polymerase III promoter sequence may further comprise a TATA element lying 5′ to said tRNA sequence or a variant or fragment thereof, the TATA element being active in said host cells; the ncRNA polynucleotide sequence may be or comprise a guideRNA (gRNA), a crRNA and a tracrRNA; and/or the RNA polymerase III terminator sequence may be or comprise a poly-T termination signal.

In some aspects, the present description relates to a vector comprising an autonomously replicating sequence (ARS) from Issatchenkia orientalis (Pichia kudriavzevii or Candida krusei), or a variant or fragment of said ARS that confers autonomously replicating activity to a vector when transformed in I. orientalis cells.

In embodiments, the ARS may comprise a nucleic acid sequence at least 60%, 65%, 70%, 75%, 80%, 85%, 90%, or 95% identical to SEQ ID NO: 1, 4, 5, 6, 7, 8, 31, and/or 32, and/or comprise at least 8, 9, 10, 11, 12, 13, 14, 15, 16, 17, 18, 19, or 20 contiguous nucleotides of any one of SEQ ID NOs: 1 and 4-8. In embodiments, the ARS may comprise a nucleic acid sequence at least 60%, 65%, 70%, 75%, 80%, 85%, 90%, or 95% identical to any one of SEQ ID NOs: 9-30, or a fragment thereof having autonomously replicating activity. In embodiments, the ARS may confer autonomously replicating activity to the vector when transformed in a yeast or fungus which is: Issatchenkia orientalis (Pichia kudriavzevii or Candida krusei), Candida ethanolica, Pichia membranifaciens, Candida intermedia, Pichia sorbitophila, Candida sorboxylosa, Scheffersomyces lignosus, Candida tanzawaensis, Scheffersomyces shehatae, Debaryomyces hansenii, Scheffersomyces stipitis, Leptosphaeria biglobosa, Spathaspora girioi, Leptosphaeria maculans, Spathaspora gorwiae, Metschnikowia australis, Spathaspora hagerdaliae, Millerozyma farinosa, Spathaspora passalidarum, Nakazawaea peltata, Sugiyamaella xylanicola, Wickerhamia fluorescens, or any combination thereof.

In some aspects, the present description relates to a vector comprising an ARS that comprises a nucleic acid sequence at least 60%, 65%, 70%, 75%, 80%, 85%, 90%, or 95% identical to SEQ ID NO: 70, 71, and/or 72, or a fragment thereof having autonomously replicating activity. In embodiments, the ARS may confer autonomously replicating activity to the vector when transformed in a yeast or fungus which is: Issatchenkia orientalis (Pichia kudriavzevii or Candida krusei), Ashbya gossypii, Candida auris, Candida intermedia, Candida orthopsilosis, Candida parapsilosis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Metschnikowia bicuspidata var. bicuspidata, Millerozyma farinosa, Pichia pastoris, Pichia sorbitophila, Saccharomycetaceae sp. ‘Ashbya aceri’, Saccharomycopsis fibuligera, Scheffersomyces stipitis, T. utilis, Tetrapisispora phaffii, Vanderwaltozyma polyspora, or any combination thereof.

In embodiments, the vectors described herein may further comprise an RNA polymerase II promoter and an RNA polymerase II terminator; an RNA polymerase III promoter and an RNA polymerase III terminator; or both. In embodiments, the RNA polymerase II promoter may comprise a nucleic acid sequence at least 60%, 65%, 70%, 75%, 80%, 85%, 90% or 95% identical to any one of SEQ ID NOs: 33-42, or a fragment thereof having RNA polymerase II promoter activity; and/or (ii) the RNA polymerase II terminator may comprise a nucleic acid sequence at least 60%, 65%, 70%, 75%, 80%, 85%, 90% or 95% identical to SEQ ID NO: 43 or 44, or a fragment thereof having RNA polymerase II terminator activity. In particularly embodiments, the RNA polymerase III promoter may be a tRNA gene or an rRNA promoter, or tRNA gene or an rRNA promoter from Issatchenkia orientalis (e.g., a RNA polymerase III promoter and/or RNA polymerase III terminator is as defined herein).

In embodiments, the vectors described herein may comprise: (i) a polynucleotide encoding a protein of interest, operably linked to the RNA polymerase II promoter and the RNA polymerase II terminator; and/or (ii) a polynucleotide encoding an ncRNA, operably linked to the RNA polymerase III promoter and the RNA polymerase III terminator. In embodiments, (i) the protein of interest is or comprises a ribonucleoprotein, an endonuclease, an RNA-guided endonuclease, a CRISPR endonuclease, a type I CRISPR endonuclease, a type II CRISPR endonuclease, a type III CRISPR endonuclease, a type IV CRISPR endonuclease, a type V CRISPR endonuclease, a type VI CRISPR endonuclease, CRISPR associated protein 9 (Cas9), Cpf1, CasX, or CasY; and/or (ii) the ncRNA is or comprises a guideRNA (gRNA), or a crRNA and a tracrRNA.

In embodiments, the vectors described herein may further comprise: (a) a yeast and/or fungal selectable marker; (b) a bacterial selectable marker; (c) a bacterial origin of replication; or (d) any combination of (a)-(c). The yeast and/or fungal selectable marker may be a positive or negative selectable marker, and/or the bacterial selectable marker is a positive or negative selectable marker. In a particular embodiment, the vector is a plasmid, such as a plasmid having a size less than 30 kb, 25 kb, 20 kb, 15 kb, 14 kb, 13 kb, 12 kb, 11 kb, 10 kb, 9 kb, 8 kb, 7 kb, 6 kb, or 5 kb.

In some aspects, the present description relates to an expression cassette comprising a polynucleotide encoding a protein of interest, operably linked to the RNA polymerase II promoter as defined herein, and/or to the RNA polymerase II terminator as defined herein. In embodiments, the RNA polymerase II promoter and/or the RNA polymerase II terminator is heterologous to the polynucleotide encoding the protein of interest.

In some aspects, the present description relates to a yeast or fungal cell comprising a recombinant DNA molecule as defined herein, a vector as defined herein, or an expression cassette as defined herein. In embodiments, the cell may be a yeast or fungal cell belonging to the species: Issatchenkia orientalis (Pichia kudriavzevii or Candida krusei), Ashbya gossypii, Candida auris, Candida ethanolica, Candida intermedia, Candida orthopsilosis, Candida parapsilosis, Candida sorboxylosa, Candida tanzawaensis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Leptosphaeria biglobosa, Leptosphaeria maculans, Metschnikowia australis, Metschnikowia bicuspidata var. bicuspidata, Millerozyma farinosa, Nakazawaea peltata, Pichia membranifaciens, Pichia pastoris, Pichia sorbitophila, Saccharomycetaceae sp. ‘Ashbya aceri’, Saccharomycopsis fibuligera, Scheffersomyces lignosus, Scheffersomyces shehatae, Scheffersomyces stipitis, Spathaspora girioi, Spathaspora gorwiae, Spathaspora hagerdaliae, Spathaspora passalidarum, Sugiyamaella xylanicola, T. utilis, Tetrapisispora phaffii, Vanderwaltozyma polyspora, or Wickerhamia fluorescens.

In some aspects, the present description relates to the use of the recombinant DNA molecule as defined herein, the vector as defined herein, or the expression cassette as defined herein, for genetically engineering host yeast or fungal cells.

In some aspects, the present description relates to the use of the recombinant DNA molecule as defined, the vector as defined herein, or the expression cassette as defined herein, for producing a product of interest from host yeast or fungal cells comprising said recombinant DNA molecule, said vector, or said expression cassette.

In some aspects, the present description relates to a method for genetically engineering host yeast or fungal cells, the method comprising transforming the host yeast or fungal cells with the recombinant DNA molecule as defined herein, the vector as defined herein, or the expression cassette as defined herein.

In some aspects, the present description relates to a method for producing a product of interest from host yeast or fungal cells, the method comprising: (a) providing the yeast or fungal cell as defined herein, wherein the yeast or fungal cell produces a product of interest; and (b) culturing said yeast or fungal cell under conditions enabling the synthesis of said product of interest. In embodiments, the product of interest referred to herein may be or comprise an organic acid, succinic acid, lactic acid, and/or malic acid.

In some aspects, the present description relates to a method for genetically engineering a yeast or fungal cell, the method comprising: (a) providing a yeast or fungal cell that has been engineered to express a genomically-integrated RNA-guided endonuclease; (b) transforming the yeast or fungal cell with: (i) an expression vector comprising a vector selection marker and a guide RNA (gRNA) operably linked to an RNA polymerase III promoter and terminator, wherein the gRNA is designed to assemble with the RNA-guided endonuclease to cleave at a genomic site of interest; and (ii) a template double-stranded DNA (dsDNA) wherein the template dsDNA is designed to direct repair or edition of the cleaved genomic DNA; and (c) culturing the transformed yeast or fungal cell in selective media and isolating a positive transformant comprising the desired genomic integration of the expression cassette. In embodiments, the method may further comprise (d) culturing the positive transformant in nonselective media, thereby allowing the positive transformant to lose the expression vector. In embodiments, the method may further comprise repeating (b) to (d) until the desired level of genetic engineering has been achieved. In embodiments, the method may further comprise (e) further transforming the positive transformant with an expression vector and template dsDNA as defined herein, which are designed to remove the genomically-integrated RNA-guided endonuclease from the genome of the yeast or fungal cell. In embodiments, the genomic selection marker may be SUC2, LEU2, TRPI, URA3, HIS3, LYS2, or MET15. In embodiments, the template dsDNA may comprise an expression cassette encoding a protein of interest operably linked to an RNA polymerase II promoter and terminator for expression in the yeast or fungal cell, wherein the template dsDNA is designed to direct repair or edition of the cleaved genomic DNA such that the expression cassette is integrated at the genomic site of interest.

### General Definitions

Headings, and other identifiers, e.g., (a), (b), (i), (ii), (I), (II), etc., are presented merely for ease of reading the specification and claims. The use of headings or other identifiers in the specification or claims does not necessarily require the steps or elements to be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the transformation of three genetically unmodified, wild and distinct I. orientalis isolates (strains 1, 2 and 3), with three plasmids each cloned with unique ARS-containing genomic DNA sequences (ARS-1, ARS-2, and ARS-3).

FIG. 2A shows the approximate positions of forward (F) and reverse (R) primer pairs (arrows) relative to the ARS-containing genomic DNA sequence ARS-1 (black line), which were used to generate overlapping amplicons.

FIG. 2B shows the results of transforming I. orientalis cells with a plasmid containing ARS-1, as compared to plasmids containing amplicons generated from the primer pairs F1+R1 (FIG. 2C) and primer pairs F3+R3 (FIG. 2D).

FIG. 3A shows the results of a nucleotide BLAST alignment using the sequence of the 90-bp amplicon produced by primer pairs F3+R3. A corresponding multiple sequence alignment is shown in FIG. 3B, and phylogenic tree analysis is shown in FIG. 3C.

FIG. 4A-4C show diagnostic PCR results of pdclA::GFP. Three I. orientalis tRNAs (threonine, FIG. 4A; leucine, FIG. 4B; and proline, FIG. 4C) were used as promoters to express a CRISPR gRNA designed to delete endogenous I. orientalis pyruvate decarboxylase isozyme 1 (IoPDC1) and replace it with a gene encoding the marker GFP. PCR was used to measure the presence of a genome integrated GFP gene to confirm genome editing. A wild type strain containing IoPDC1+ wild type control is on the far right in FIG. 4C. The “A” symbol represents a PCR reaction in which an external primer is paired with an internal GFP primer, and “wt” represents a PCR reaction in which an external primer is paired with an internal IoPDC1 primer. The correct integration of the GFP cassette was 100% for each tRNA used.

FIG. 5 shows the taxonomic results of a BLAST analysis of the I. orientalis genomic DNA fragment ARS-2 (SEQ ID NO: 2).

FIG. 6 is a multiple sequence alignment of the validated I. orientalis tRNA sequences of SEQ ID NOs: 45-47. Shaded in black are two highly conserved regions (SEQ ID NOs: 66 and 67), which may function as I. orientalis box A and box B RNA polymerase III transcriptional control sequences.

FIG. 7 shows a summary of pairwise nucleic acid sequence similarity scores between each of the I. orientalis tRNA sequences listed in Table 3 (SEQ ID NOs: 45-60) generated using CLUSTALW alignment tool.

### SEQUENCE LISTING

This application contains a Sequence Listing in computer readable form entitled Sequence_Listing.txt, created May 9, 2018 having a size of about 31 kb. The computer readable form is incorporated herein by reference.

| SEQ | |
|---|---|
| ID NO: | Description |
| 1 | I. orientalis cloned genomic DNA fragment containing ARS-1 |
| 2 | I. orientalis cloned genomic DNA fragment containing ARS-2 |
| 3 | [Skipped sequence] |
| 4 | 90-bp fragment of SEQ ID NO: 1 sufficient to confer |
| | autonomously replicating activity |
| 5 | Conserved 45-bp subfragment of SEQ ID NO: 4 |
| 6 | Consensus sequence for ARS-1 |
| 7 | Consensus sequence for ARS-1 |
| 8 | Highly conserved 18-bp subfragment of ARS-1 |
| 9 | Genomic DNA fragment from Candida ethanolica |
| 10 | Genomic DNA fragment from Candida intermedia |
| 11 | Genomic DNA fragment from Candida sorboxylosa |
| 12 | Genomic DNA fragment from Candida tanzawaensis |
| 13 | Genomic DNA fragment from Debaryomyces hansenii |
| 14 | Genomic DNA fragment from Leptosphaeria biglobosa |
| 15 | Genomic DNA fragment from Leptosphaeria maculans |
| 16 | Genomic DNA fragment from Metschnikowia australis |
| 17 | Genomic DNA fragment from Millerozyma farinose |
| 18 | Genomic DNA fragment from Nakazawaea peltata |
| 19 | Genomic DNA fragment from Pichia kudriayzevii |
| 20 | Genomic DNA fragment from Pichia membranifaciens |
| 21 | Genomic DNA fragment from Pichia sorbitophila |
| 22 | Genomic DNA fragment from Scheffersomyces lignosus |
| 23 | Genomic DNA fragment from Scheffersomyces shehatae |
| 24 | Genomic DNA fragment from Scheffersomyces stipitis |
| 25 | Genomic DNA fragment from Spathaspora girioi |
| 26 | Genomic DNA fragment from Spathaspora gorwiae |
| 27 | Genomic DNA fragment from Spathaspora hagerdaliae |
| 28 | Genomic DNA fragment from Spathaspora passalidarum |
| 29 | Genomic DNA fragment from Sugiyamaella xylanicola |
| 30 | Genomic DNA fragment from Wickerhamia fluorescens |
| 31 | Consensus sequence from alignment of SEQ ID NOs: 9-30 |
| 32 | Consensus sequence from alignment of SEQ ID NOs: 9-30 |
| 33 | I. orientalis TEF1 Promoter |
| 34 | I. orientalis TDH3 Promoter |
| 35 | I. orientalis PGK1 Promoter |
| 36 | I. orientalis PGI1 Promoter |
| 37 | I. orientalis PFK1 Promoter |
| 38 | I. orientalis PDC1 Promoter |
| 39 | I. orientalis HHF1 Promoter |
| 40 | I. orientalis ENO1 Promoter |
| 41 | I. orientalis CCW12 Promoter |
| 42 | I. orientalis ACT1 Promoter |
| 43 | I. orientalis ADH1 Terminator |
| 44 | I. orientalis TDH3 Terminator |
| 45 | I. orientalis tRNA Threonine |
| 46 | I. orientalis tRNA Leucine |
| 47 | I. orientalis tRNA Proline |
| 48 | I. orientalis tRNA Methionine |
| 49 | I. orientalis tRNA Glutamine |
| 50 | I. orientalis tRNA Glutamate |
| 51 | I. orientalis tRNA Valine |
| 52 | I. orientalis tRNA Serine |
| 53 | I. orientalis tRNA Histidine |
| 54 | I. orientalis tRNA Phenylalanine |
| 55 | I. orientalis tRNA Arginine |
| 56 | I. orientalis tRNA Alanine |
| 57 | I. orientalis tRNA Isoleucine |
| 58 | I. orientalis tRNA Asparagine |
| 59 | I. orientalis tRNA Cysteine |
| 60 | I. orientalis tRNA Tryptophan |
| 61 | I. orientalis tRNA Threonine (SEQ ID NO: 45) + |
| | ~100-bp 5′ genomic DNA sequence |
| 62 | I. orientalis tRNA Leucine (SEQ ID NO: 46) + |
| | ~100-bp 5′ genomic DNA sequence |
| 63 | I. orientalis tRNA Proline (SEQ ID NO: 47) + |
| | ~100-bp 5′ genomic DNA sequence |
| 64 | S. cerevisiae tRNA Tyrosine |
| 65 | S. cerevisiae tRNA Phenylalanine |
| 66 | I. orientalis tRNA consensus sequence TGGnCnAGT |
| 67 | I. orientalis tRNA consensus sequence GTTCnAnnC |
| 68 | I. orientalis tRNA consensus sequence GnTCnAnnC |
| 69 | I. orientalis tRNA consensus sequence GTTCnAnnC |
| 70 | Consensus sequence for ARS-2 |
| 71 | Consensus sequence for ARS-2 |
| 72 | Consensus sequence for ARS-2 |

### DETAILED DESCRIPTION

The present description relates to genetic tools and methods to facilitate transformation/genome editing/genetic engineering of industrially-useful yeast/fungal species, such as Issatchenkia orientalis, for which a robust set of genetic tools, such as stably inherited and maintained plasmids and functional control sequences is presently lacking. In fact, genetic tools developed and optimized for model organisms such as S. cerevisiae simply do not function in many industrially useful yeast/fungal extremophiles, rendering the engineering of these organisms as difficult, laborious, and time-intensive processes. Thus, there is a need for novel genetic tools and methods to facilitate the genomic engineering of industrially useful extremophiles such as I. orientalis. More specifically, autonomously replicating sequences (ARSs), RNA polymerase II and III promoters, RNA polymerase II and III terminators, expression cassettes, and vectors comprising same are described herein, as well as uses and methods relating to same.

### Autonomously Replicating Sequences

In some embodiments, the present description relates to one or more autonomously replicating sequences. As used herein, an “autonomously replicating sequence” or “ARS” refers to a sequence that has or can confer autonomously replicating activity to a nucleic acid molecule that is delivered intracellularly to a fungal or yeast cell of interest (e.g., an industrially useful yeast species such as I. orientalis). An ARS generally contains a yeast or fungal origin of replication, which may include a conserved consensus sequence that may function as a binding site for the Origin Recognition Complex (ORC), as well as flanking regions which may positively influence the vector's ability to autonomously replicate. In some embodiments, the ARS may be of any length, but is typically between 30 and 500 bp, but may be between 30, 35, 40, 45, 50, 55, 60, 65, 70, 75, or 80 bp and 90, 100, 120, 140, 160, 180, 200, 250, 300, 350, 400, 450 or 500 bp.

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the sequence of SEQ ID NO: 1 or 2 (referred to herein as ARS-1 and ARS-2, respectively), or a fragment thereof sufficient to confer autonomously replicating activity (e.g., in a yeast of fungal cell of interest). These sequences correspond to I. orientalis genomic DNA fragments that are sufficient to confer autonomously replicating activity when comprised in a plasmid expressed in an I. orientalis host cell, as described herein in Examples 1 and 2. These sequences correspond to independent, non-overlapping I. orientalis genomic DNA fragments identified using a restriction enzyme-based shotgun cloning approach.

### ARS-1

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the consensus sequence of SEQ ID NO: 6 or 7, or a fragment thereof having autonomously replicating activity (i.e., a fragment that, when comprised in a vector or extra-chromosomal DNA, can confer to the vector or extra-chromosomal DNA the ability to autonomously replicate in a host cell of interest). These consensus sequences were identified via bioinformatic analyses of over 1000 genomic DNA sequences from over 145 unique species, using a genomic DNA fragment from an I. orientalis host cell (ARS-1) sufficient to confer autonomously replicating activity.

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the sequence of any one of SEQ ID NOs: 4 or 5, or a fragment thereof sufficient to confer autonomously replicating activity (e.g., in a yeast of fungal cell of interest). SEQ ID NO: 4 corresponds to a 90-bp fragment of SEQ ID NO: 1 (ARS-1) that is shown herein to be sufficient to confer autonomously replicating activity when comprised in a plasmid expressed in an I. orientalis host cell. SEQ ID NO: 5 corresponds to a 45-bp subfragment of SEQ ID NO: 4 that is particularly conserved across multiple yeast or fungal strains.

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the sequence of any one of SEQ ID NOs: 9-30, or a fragment thereof sufficient to confer autonomously replicating activity (e.g., in a yeast of fungal cell of interest). These sequences correspond to genomic DNA fragments from different yeast or fungal species identified based on their relatively high sequence identity to SEQ ID NO: 4.

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the consensus sequence of SEQ ID NO: 31 or 32, or a fragment thereof sufficient to confer autonomously replicating activity (e.g., in a yeast of fungal cell of interest). These consensus sequences were identified via a multiple sequence alignment of SEQ ID NOs: 9-30.

In some embodiments, the ARSs described herein may comprise a nucleic acid sequence at least 80%, 81%, 82%, 83%, 84%, 85%, 86%, 87%, 88%, 89%, 90%, 91%, 92%, 93%, 94%, 95%, 96%, 97%, 98% or 99% identical to the sequence of SEQ ID NO: 8. This sequence corresponds to an 18-bp fragment of SEQ ID NOs: 1, 4, 5, and 9-30, which was identified as being highly conserved (e.g., at least 99% identical) in over 1000 genomic DNA sequences analyzed from over 145 unique species.

In some embodiments, the ARSs described herein may comprise at least 8, 9, 10, 11, 12, 13, 14, 15, 16, 17, 18, 19, or 20 contiguous nucleotides of any one of SEQ ID NOs: 1 and 4-8.

In some embodiments, the ARSs described herein may confer autonomously replicating activity to a nucleic acid expressed in a yeast or fungus of the genus: Issatchenkia, Pichia, Candida krusei, Scheffersomyces, Debaryomyces, Leptosphaeria, Spathaspora, Metschnikowia, Millerozyma, Nakazawaea, Sugiyamaella, Wickerhamia, or any combination thereof. In some embodiments, the ARSs described herein may confer autonomously replicating to a nucleic acid expressed in a yeast or fungus of the species: Issatchenkia orientalis (Pichia kudriavzevii or Candida krusei), Candida ethanolica, Pichia membranifaciens, Candida intermedia, Pichia sorbitophila, Candida sorboxylosa, Scheffersomyces lignosus, Candida tanzawaensis, Scheffersomyces shehatae, Debaryomyces hansenii, Scheffersomyces stipitis, Leptosphaeria biglobosa, Spathaspora girioi, Leptosphaeria maculans, Spathaspora gorwiae, Metschnikowia australis, Spathaspora hagerdaliae, Millerozyma farinosa, Spathaspora passalidarum, Nakazawaea peltata, Sugiyamaella xylanicola, Wickerhamia fluorescens, or any combination thereof.

As used herein unless specified otherwise, the expression “I. orientalis” is intended to include all currently accepted forms and/or synonyms of this species, which include Pichia kudriavzevii or Candida krusei (anamorph or asexual form) (Kurtzman et al., 1980; Kurtzman et al., 2010).

### ARS-2

In some embodiments, the ARSs described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the consensus sequence of SEQ ID NO: 70, 71, and/or 72, or a fragment thereof having autonomously replicating activity.

The nucleic acid sequence set forth in SEQ ID NO: 70 corresponds to a 73-bp consensus sequence identified of ARS-2 (SEQ ID NO: 2), which was highly conserved (over 85% sequence identity) across multiple species, suggesting cross-species ARS functionality. Accordingly, in some embodiments, the ARSs described herein may confer autonomously replicating activity to a nucleic acid expressed in a yeast or fungus of the genus: Ashbya, Candida, Cyberlindnera, Debaryomyces, Eremothecium, Kluyveromyces, Komagataella, Komagataella, Lachancea, Metschnikowia, Millerozyma, Pichia, Saccharomycetaceae, Saccharomycopsis, Scheffersomyces, T. utilis, Tetrapisispora, Vanderwaltozyma polyspora, or any combination thereof. In some embodiments, the ARSs described herein may confer autonomously replicating to a nucleic acid expressed in a yeast or fungus of the species: Ashbya gossypii, Candida auris, Candida intermedia, Candida orthopsilosis, Candida parapsilosis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Metschnikowia bicuspidata var. bicuspidata, Millerozyma farinosa, Pichia kudriavzevii (I. orientalis), Pichia pastoris, Pichia sorbitophila, Saccharomycetaceae sp. ‘Ashbya aceri’, Saccharomycopsis fibuligera, Scheffersomyces stipitis, T. utilis, Tetrapisispora phaffii, Vanderwaltozyma polyspora, or any combination thereof.

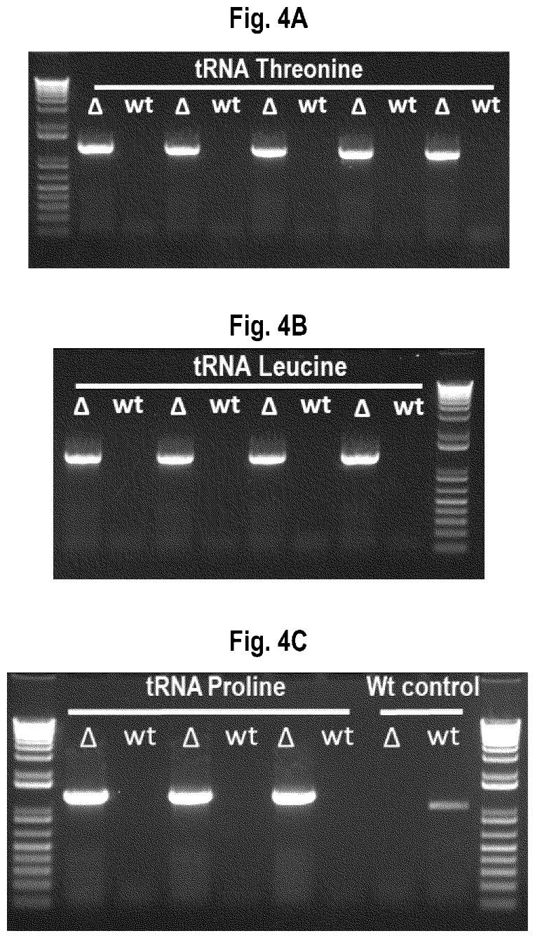

The nucleic acid sequence set forth in SEQ ID NO: 71 corresponds to a consensus sequence found in 17 different genomic DNA database entries from Pichia kudriavzevii (I. orientalis), including different entries on each of Pichia kudriavzevii chromosomes 1-8 (see FIG. 5). Interestingly, both SEQ ID NOs: 70 and 71 were found to contain a 17-bp fragment set forth as SEQ ID NO: 72, which was 100% conserved in all the foregoing species as well as a plurality of other fungal species.

In some embodiments, the ARSs described herein may comprise at least 8, 9, 10, 11, 12, 13, 14, 15, 16, 17, 18, 19, or 20 contiguous nucleotides of any one of SEQ ID NOs: 2 and 70-72.

### Promoters, Terminators, and Expression Cassettes

In some embodiments, the present description relates to promoters and/or terminators that may be useful for expressing a polynucleotide of interest in a yeast or fungal cell of interest (e.g., a yeast of the genus Issatchenkia such as I. orientalis).

As used herein, a “promoter” refers to any nucleic acid sequence that regulates the initiation of transcription for a polynucleotide under its control. A promoter minimally includes the genetic elements necessary for the initiation of transcription (e.g., RNA polymerase II- or III-mediated transcription), and may further include one or more genetic elements that serve to specify the prerequisite conditions for transcriptional initiation. A promoter may be encoded by the endogenous genome of a host cell, or it may be introduced as part of a recombinantly engineered polynucleotide. A promoter sequence may be taken from one host species and used to drive expression of a gene in a host cell of a different species. As used herein, a “terminator” refers to any nucleotide sequence that is sufficient to terminate a transcript transcribed by RNA polymerase II or III.

### RNA Polymerase II Promoters and Terminators

In some embodiments, promoters described herein may include RNA polymerase II promoters, preferably having RNA polymerase II promoter activity in I. orientalis. In some embodiments, the RNA polymerase II promoters described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 33-42, or a fragment thereof having RNA polymerase II promoter activity, preferably in I. orientalis.

In some embodiments, terminators described herein may include RNA polymerase II terminators, having RNA polymerase II terminator activity in I. orientalis. In some embodiments, the RNA polymerase II terminators described herein may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 43 or 44, or a fragment thereof having RNA polymerase II terminator activity, preferably in I. orientalis.

In some embodiments, the RNA polymerase II promoters and RNA polymerase II terminators described herein may be operably linked to a polynucleotide encoding a protein of interest to be expressed in a yeast or fungal cell of interest (e.g., I. orientalis). In some embodiments, the protein of interest is or comprises an endonuclease, an RNA-guided endonuclease, a CRISPR endonuclease, a type I CRISPR endonuclease, a type II CRISPR endonuclease, a type III CRISPR endonuclease, a type IV CRISPR endonuclease, a type V CRISPR endonuclease, a type VI CRISPR endonuclease, CRISPR associated protein 9 (Cas9), Cpf1, CasX, or CasY (Burstein et al., 2017).

### RNA Polymerase III Promoters and Terminators

Unlike RNA polymerase II, RNA polymerase III transcribes DNA to synthesize RNA molecules that do not encode a polypeptide translated/expressed by the cell (e.g. ribosomal 5S rRNA, tRNA and other small RNAs). As used herein, RNA molecules that do not encode a polypeptide to be translated/expressed in a host cell are referred to interchangeably herein as “non-polypeptide-coding RNA”, “non-coding RNA”, or “ncRNA”. For greater clarity, as used herein, a polynucleotide or gene that encodes an ncRNA refers to the fact that the polynucleotide is transcribed (or is transcribable) into a functional ncRNA molecule. Such polynucleotides or genes are referred to herein as a “ncRNA polynucleotide” or “ncRNA gene”.

Endogenous RNA polymerase III can be utilized to transcribe functional ncRNA molecules in vivo by introducing into a host cell an expression cassette containing a recombinant polynucleotide encoding the ncRNA under the control of an RNA polymerase III promoter. As used herein, an “RNA polymerase III promoter” refers to a nucleotide sequence that directs the transcription of RNA by RNA polymerase III. RNA polymerase III promoters may include a full-length promoter or a fragment thereof sufficient to drive transcription by RNA polymerase III, as well as other control elements (e.g., TATA elements) that are required for transcription. A general description of RNA polymerase III promoters can be found in Schramm and Hernandez, 2002.

In some cases, the DNA sequences of transfer RNA (tRNA) genes may be employed as RNA polymerase III promoters, with some transcriptional control sequences (e.g., TATA elements) being upstream of the tRNA transcriptional start site, and other control elements (e.g., box A and box B sequences) being intragenic (i.e., within the tRNA gene sequence itself). More specifically, tRNA sequences may be operably linked to a polynucleotide encoding an ncRNA of interest in order to drive in vivo transcription of the ncRNA. Unfortunately, standard molecular cloning tools and control sequences that function in traditional yeasts such as S. cerevisiae may not be operable in non-traditional species such as I. orientalis, which are generally regarded as being more difficult to work with. Indeed, initial attempts at utilizing S. cerevisiae tRNA sequences, such as S. cerevisiae tRNA Tyrosine (SEQ ID NO: 64) and S. cerevisiae tRNA Phenylalanine (SEQ ID NO: 65) failed at expressing ncRNA in I. orientalis. Thus, extensive work was performed to interrogate I. orientalis genomic DNA sequences to identify, clone and validate tRNA sequences that may function as RNA polymerase III promoters in I. orientalis, as described herein in Examples 4 and 5.

Accordingly, in some aspects, the present description relates to recombinant DNA molecules useful for expressing ncRNA in host cells (e.g., yeast or fungal cells). The recombinant DNA molecules generally comprise an expression cassette having an RNA polymerase III promoter sequence, a polynucleotide sequence encoding an ncRNA to be expressed in the host cells, and an RNA polymerase III terminator sequence, wherein the RNA polymerase III promoter and terminator sequences enable transcription of the ncRNA polynucleotide when introduced into the host cells.

In some embodiments, the RNA polymerase III promoter sequence may comprise a tRNA sequence derived from I. orientalis genomic DNA, or a variant or fragment of the tRNA sequence having/retaining RNA polymerase III promoter activity, preferably in at least I. orientalis cells. In some embodiments, the RNA polymerase III promoters defined herein may include a tRNA sequence (e.g., an I. orientalis-derived tRNA sequence) for arginine, histidine, lysine, aspartate, glutamate, serine, threonine, asparagine, glutamine, cysteine, glycine, proline, alanine, isoleucine, leucine, methionine, phenylalanine, tryptophan, tyrosine, or valine; or a variant or fragment thereof having/retaining RNA polymerase III promoter activity, preferably in at least I. orientalis cells.

In some embodiments, the tRNA sequence, or variant or fragment thereof described herein, may comprise the I. orientalis tRNA consensus sequence of SEQ ID NO: 66, 67, 68 or 69, which may relate to control elements (e.g., box A or box B) required for RNA polymerase III transcription.

In some embodiments, the tRNA sequence, or variant or fragment thereof described herein, may comprise a nucleic acid sequence at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 45-63, or a fragment thereof having RNA polymerase III promoter activity, preferably in I. orientalis cells.

In some embodiments, the tRNA sequence, or variant or fragment thereof described herein, may comprise at least 8, 9, 10, 11, 12, 13, 14, 15, 16, 17, 18, 19, 20, 21, 22, 23, 24, 25, 26, 27, 28, 29, or 30 contiguous nucleotides of any one of SEQ ID NOs: 45-63.

In some embodiments, the RNA polymerase III promoters defined herein may include a ribosomal RNA (rRNA) gene or sequence (e.g., a 5S rRNA), preferably derived from I. orientalis genomic DNA.

In some embodiments, the RNA polymerase III terminators described herein may comprise a poly-T or T-rich stretch (e.g., comprising at least 4-6 consecutive T nucleotides).

In some embodiments, the RNA polymerase III promoters and RNA polymerase III terminators described herein may be operably linked to a polynucleotide encoding a ncRNA (a ncRNA polynucleotide). Examples of ncRNAs of interest may include smallRNA (sRNA), non-protein-coding RNA (npcRNA), non-messenger RNA (nmRMA), functional RNA (fRNA), microRNA (miRNA), small interfering RNA (siRNA), guideRNA (gRNA), crRNA and tracrRNA. In some embodiments, the ncRNA polynucleotides described herein may include RNA components of functional ribonucleoproteins, such as a guideRNA (gRNA), a crRNA, and a tracrRNA (e.g., for use with an RNA-guided endonuclease such as a CRISPR endonuclease, a type I CRISPR endonuclease, a type II CRISPR endonuclease, a type III CRISPR endonuclease, a type IV CRISPR endonuclease, a type V CRISPR endonuclease, a type VI CRISPR endonuclease, CRISPR associated protein 9 (Cas9), Cpf1, CasX, or CasY (Burstein et al., 2017)). Such ncRNAs may be employed, along with other ARS and control sequences described herein, to greatly facilitate genetic engineering host cells of industrially useful yeast or fungal cells, such as the ones mentioned herein.

In some embodiments, the present description relates to an expression cassette comprising one or more of the promoters and/or terminators described herein. In some embodiments, the expression cassette may comprise a polynucleotide encoding a protein of interest, operably linked to the RNA polymerase II promoter as described herein and an RNA polymerase II terminator as described herein. In some embodiments, the RNA polymerase II promoter and/or the RNA polymerase II terminator may be heterologous to the polynucleotide encoding the protein of interest.

In some embodiments, the expression cassette may comprise an ncRNA polynucleotide, operably linked to the RNA polymerase III promoter as described herein, and to an RNA polymerase III terminator as described herein. In some embodiments, the ncRNA polynucleotide may be heterologous to the RNA polymerase III promoter and/or RNA polymerase III terminator. In some embodiments, the expression cassette is non-native, meaning that it is not found in the genomic DNA of a non-genetically modified organism (e.g., a wild-type strain of yeast or fungus). In some embodiments, the expression cassette, RNA polymerase III promoter, RNA polymerase III terminator, and/or the ncRNA polynucleotide, is/are non-native, exogenous, or heterologous with respect to the host yeast or fungal cells. In some embodiments, the ncRNA polynucleotide is heterologous with respect to the RNA polymerase III promoter and/or RNA polymerase III terminator.

### Hybridization Polynucleotides

In some embodiments, the present description relates to polynucleotides that hybridize to the complement of any one of SEQ ID NOs: 1, 2, 4-63, or 70-72. Hybridization under stringent conditions is preferred, which may include hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions may also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. may be employed. Yet additional stringent hybridization conditions may include hybridization at 60° C., or higher, and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

### Vectors and Cells

In some embodiments, the present description relates to vectors comprising one or more of the ARSs described herein. As used herein, a “vector” refers to a DNA construct that is capable of delivering, and preferably expressing, one or more polynucleotides of interest in a host cell (e.g., yeast or fungal cell). In some embodiments, the vectors described herein may be a plasmid, such as an episomal plasmid (e.g., a 2-micron plasmid), a yeast replicating plasmid (YRp), or a yeast centromere plasmid (YCp). In some embodiments, the vectors described herein may be a yeast artificial chromosome (YAC). In some embodiments, the plasmid may have a size less than 30 kb, 25 kb, 20 kb, 15 kb, 14 kb, 13 kb, 12 kb, 11 kb, 10 kb, 9 kb, 8 kb, 7 kb, 6 kb, or 5 kb. Smaller plasmids may advantageously provide higher transformation efficiency.

In some embodiments, the vectors described herein may further comprise a yeast and/or fungal selection marker (e.g., an I. orientalis selection marker), which can be a positive or a negative selection marker. Examples of yeast selection markers include SUC2, LEU2, TRPI, URA3, HIS3, LYS2, and MET15. In some embodiments, the selection marker may be an antibiotic resistant gene such as NatR and/or HpH, which confer resistance to the antibiotics nourseothricin and hygromycin, respectively. For example, I. orientalis was found to be sensitive to nourseothricin concentrations at or exceeding 100 mg/L and hygromycin concentrations at or exceeding 400 mg/L.

In some embodiments, the vectors described herein may further comprise a bacterial origin of replication. In some embodiments, the vectors described herein may further comprise a bacterial selection marker, which can be a positive or negative selection marker, such as an antibiotic resistance gene.

In some embodiments, the present description further relates to host cells (e.g., a yeast or fungal cell) that (stably) comprise or are (stably) transformed with a vector or expression cassette as described herein. In some embodiments, the host cell may be of the genus: Issatchenkia, Pichia, Candida krusei, Scheffersomyces, Debaryomyces, Leptosphaeria, Spathaspora, Metschnikowia, Millerozyma, Nakazawaea, Sugiyamaella, or Wickerhamia. In some embodiments, the host cell may be of the species: Issatchenkia orientalis (Pichia kudriavzevii or Candida krusei), Candida ethanolica, Pichia membranifaciens, Candida intermedia, Pichia sorbitophila, Candida sorboxylosa, Scheffersomyces lignosus, Candida tanzawaensis, Scheffersomyces shehatae, Debaryomyces hansenii, Scheffersomyces stipitis, Leptosphaeria biglobosa, Spathaspora girioi, Leptosphaeria maculans, Spathaspora gorwiae, Metschnikowia australis, Spathaspora hagerdaliae, Millerozyma farinosa, Spathaspora passalidarum, Nakazawaea peltata, Sugiyamaella xylanicola, or Wickerhamia fluorescens.

### Genetic Engineering

In some embodiments, the present description further relates to the use of a vector or expression cassette as described herein for genetically engineering a yeast or a fungal cell. In some embodiments, the present description further relates to the use of a vector or expression cassette as described herein for producing a product of interest (e.g., an organic acid such as succinic acid), from a yeast or fungal cell comprising said vector or expression cassette.

In some embodiments, the present description further relates to a method for genetically engineering a yeast or a fungal cell, the method comprising transforming the yeast or fungus with a vector or expression cassette as described herein.

In some embodiments, the present description further relates to a method for producing a product of interest from a yeast or fungal cell, the method comprising providing a yeast or fungal cell as described herein, wherein the yeast or fungal cell produces a product of interest; and culturing the yeast or fungal cell under conditions enabling the synthesis of the product of interest (e.g., an organic acid such as succinic acid, lactic acid, or malic acid).

In some embodiments, the present description relates to a method for genetically engineering a yeast or fungal cell to express a genomically-integrated RNA-guided endonuclease. The RNA-guided endonuclease may be integrated into the genome of the yeast or fungal cell using one or more of the vectors and/or expression cassettes described herein. For example, the RNA-guided endonuclease may be integrated into the genome of the yeast or fungal cell by transforming the cell with an expression vector (e.g., plasmid) comprising: (a) a polynucleotide encoding the RNA-guided endonuclease (e.g., Cas9, Cpf1, CasX, CasY, or another endonuclease herein described or known in the art), which is operably linked to an RNA polymerase II promoter and terminator; and (b) a polynucleotide that gives rise to a guide RNA (gRNA, which may include a single guide RNA (sgRNA), or a crRNA and trRNA pair), operably linked to an RNA polymerase III promoter and terminator. The transformation may include a double-stranded DNA (dsDNA) expression cassette which encodes the RNA-guided endonuclease to be inserted into the genome of the yeast or fungal cell, which serves as a DNA repair template. Following transformation, the guide RNA complexes with the vector-expressed endonuclease within the transformed cell to direct cleavage of genomic DNA at a site of interest. The DNA repair template then directs repair of cleaved genomic DNA via homologous recombination, ultimately resulting in the targeted insertion of the RNA-guided endonuclease into the genome of the yeast or fungal cell. In some embodiments, the RNA-guided endonuclease may be inserted into a genomic selection marker (e.g., URA3), thereby disrupting the marker and enabling the use of selection medium (5-fluoroorotic acid (5-FOA) medium). For yeast or fungal strains that are multiploid (e.g., diploid), the host may be homozygous for the RNA-guided endonuclease genomic insertion. In some embodiments, a single copy of the disrupted genomic selection marker (e.g., URA3) may be restored, thereby engineering a prototrophic, heterozygous (e.g., URA3/endonuclease) strain.

In some embodiments, the present description relates to a method for genetically engineering a yeast or fungal cell by providing a yeast or fungal cell that has a genomically-integrated RNA-guided endonuclease. The method may comprise transforming the yeast or fungal cell with: (i) an expression vector comprising a vector selection marker and a guide RNA (gRNA) operably linked to an RNA polymerase III promoter and terminator, wherein the gRNA is designed to assemble with the RNA-guided endonuclease to cleave at a genomic site of interest; and (ii) a template double-stranded DNA (dsDNA), wherein the template dsDNA is designed to direct repair or edition of the cleaved genomic DNA. The transformed cells may then be cultured in vector-selective media, thereby isolating positive transformants comprising the desired genomic integration of the expression cassette. In some embodiments, the template dsDNA may comprise an expression cassette encoding a protein of interest (e.g., operably linked to an RNA polymerase II promoter and terminator) for expression in the yeast or fungal cell, wherein the template dsDNA is designed to direct repair or edition of the cleaved genomic DNA such that the expression cassette is integrated at the genomic site of interest.

In some embodiments, the method may further comprise (d) culturing the positive transformant in nonselective media, thereby allowing the positive transformant to lose the expression vector. The method may further comprise repeating (b) to (d) until the desired level of genetic engineering has been achieved, and optionally (e) further transforming the positive transformant with an expression vector and repair dsDNA designed to remove the genomically-integrated RNA-guided endonuclease from the genome of the yeast or fungal cell.

### Items

In other aspects, the present description may relate to one or more of the following items:
1. A recombinant DNA molecule for expressing a non-polypeptide-encoding RNA (ncRNA) in host yeast or fungal cells, the recombinant DNA molecule comprising an expression cassette comprising: (i) an RNA polymerase III promoter sequence comprising a tRNA sequence from Issatchenkia orientalis (Pichia kudriavzevii or Candida krusei), or a variant or fragment of said tRNA sequence having RNA polymerase III promoter activity in I. orientalis cells; (ii) an ncRNA polynucleotide sequence encoding the ncRNA to be expressed in the host yeast or fungal cells; and (iii) an RNA polymerase III terminator sequence, wherein the RNA polymerase III promoter and terminator sequences enable transcription of said ncRNA polynucleotide when introduced into the host yeast or fungal cells, and wherein the expression cassette is non-native, exogenous, or heterologous with respect to the host yeast or fungal cells, and/or the ncRNA polynucleotide is heterologous with respect to the RNA polymerase III promoter and/or RNA polymerase III terminator.
2. The recombinant DNA molecule of item 1, wherein said tRNA sequence, or said variant or fragment thereof, comprises the consensus sequence of SEQ ID NO: 68 or 69.
3. The recombinant DNA molecule of item 1 or 2, wherein said tRNA sequence, or said variant or fragment thereof, comprises the consensus sequence of SEQ ID NOs: 66 and 67.
4. The recombinant DNA molecule of any one of items 1 to 3, wherein said tRNA sequence, or said variant or fragment thereof, is at least 60%, 65%, 70%, 75%, 80%, 85%, 90%, 95%, or 99% identical to any one of SEQ ID NOs: 45-63.
5. The recombinant DNA molecule of any one of items 1 to 4, wherein: (i) said RNA polymerase III promoter sequence further comprises a TATA element lying 5′ to said tRNA sequence or a variant or fragment thereof, the TATA element being active in said host cells; (ii) said ncRNA polynucleotide sequence is or comprises a guideRNA (gRNA), a crRNA and a tracrRNA; and/or (iii) said RNA polymerase III terminator sequence is or comprises a poly-T termination signal.
6. A vector comprising an autonomously replicating sequence (ARS), wherein:
   (I) the ARS comprises:
      (a) a nucleic acid sequence at least 60%, 65%, 70%, 75%, 80%, 85%, 90%, or 95% identical to SEQ ID NO: 6, or a fragment thereof having autonomously replicating activity;
      (b) a nucleic acid sequence at least 60%, 65%, 70%, 75%, 80%, 85%, 90%, or 95% identical to SEQ ID NO: 7, or a fragment thereof having autonomously replicating activity;
      (c) a nucleic acid sequence at least 60%, 65%, 70%, 75%, 80%, 85%, 90%, or 95% identical to SEQ ID NO: 31, or a fragment thereof having autonomously replicating activity;
      (d) a nucleic acid sequence at least 60%, 65%, 70%, 75%, 80%, 85%, 90%, or 95% identical to SEQ ID NO: 32, or a fragment thereof having autonomously replicating activity;
      (e) a nucleic acid sequence at least 60%, 65%, 70%, 75%, 80%, 85%, 90%, or 95% identical to SEQ ID NO: 5, or a fragment thereof having autonomously replicating activity;
      (f) a nucleic acid sequence at least 60%, 65%, 70%, 75%, 80%, 85%, 90%, or 95% identical to SEQ ID NO: 4, or a fragment thereof having autonomously replicating activity;
      (g) a nucleic acid sequence at least 80%, 85%, 90%, or 95% identical to SEQ ID NO: 8;
      (h) a nucleic acid sequence at least 60%, 65%, 70%, 75%, 80%, 85%, 90%, or 95% identical to SEQ ID NO: 1, or a fragment thereof having autonomously replicating activity;
      (i) at least 8, 9, 10, 11, 12, 13, 14, 15, 16, 17, 18, 19, or 20 contiguous nucleotides of any one of SEQ ID NOs: 1 and 4-8; or
      (j) any combination of (a)-(i); or
   (II) the ARS comprises a nucleic acid sequence at least 60%, 65%, 70%, 75%, 80%, 85%, 90%, or 95% identical to SEQ ID NO: 70, 71, and/or 72, or a fragment thereof having autonomously replicating activity.
7. The vector of item 6 comprising:
   the ARS of (I), wherein said ARS confers autonomously replicating activity to the vector when transformed in a yeast or fungus which is: Issatchenkia orientalis (Pichia kudriavzevii or Candida krusei), Candida ethanolica, Pichia membranifaciens, Candida intermedia, Pichia sorbitophila, Candida sorboxylosa, Scheffersomyces lignosus, Candida tanzawaensis, Scheffersomyces shehatae, Debaryomyces hansenii, Scheffersomyces stipitis, Leptosphaeria biglobosa, Spathaspora girioi, Leptosphaeria maculans, Spathaspora gorwiae, Metschnikowia australis, Spathaspora hagerdaliae, Millerozyma farinosa, Spathaspora passalidarum, Nakazawaea peltata, Sugiyamaella xylanicola, Wickerhamia fluorescens, or any combination thereof; or the ARS of (II), wherein said ARS confers autonomously replicating activity to the vector when transformed in a yeast or fungus which is: Issatchenkia orientalis (Pichia kudriavzevii or Candida krusei), Ashbya gossypii, Candida auris, Candida intermedia, Candida orthopsilosis, Candida parapsilosis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Metschnikowia bicuspidata var. bicuspidata, Millerozyma farinosa, Pichia pastoris, Pichia sorbitophila, Saccharomycetaceae sp. ‘Ashbya aceri’, Saccharomycopsis fibuligera, Scheffersomyces stipitis, T. utilis, Tetrapisispora phaffii, Vanderwaltozyma polyspora, or any combination thereof.
8. The vector of item 6 or 7, wherein the ARS comprises a nucleic acid sequence at least 60%, 65%, 70%, 75%, 80%, 85%, 90%, or 95% identical to any one of SEQ ID NOs: 9-30, or a fragment thereof having autonomously replicating activity.
9. The vector of any one of items 6 to 8, further comprising: (i) a promoter and/or a terminator; (ii) an RNA polymerase II promoter and an RNA polymerase II terminator; (iii) an RNA polymerase III promoter and an RNA polymerase III terminator; or (iv) both (ii) and (iii).
10. The vector of item 9, wherein: (i) the RNA polymerase II promoter comprises a nucleic acid sequence at least 60%, 65%, 70%, 75%, 80%, 85%, 90% or 95% identical to any one of SEQ ID NOs: 33-42, or a fragment thereof having RNA polymerase II promoter activity; and/or (ii) the RNA polymerase II terminator comprises a nucleic acid sequence at least 60%, 65%, 70%, 75%, 80%, 85%, 90% or 95% identical to SEQ ID NO: 43 or 44, or a fragment thereof having RNA polymerase II terminator activity.
11. The vector of item 9 or 10, wherein the RNA polymerase III promoter is a tRNA gene or an rRNA promoter, or tRNA gene or an rRNA promoter from Issatchenkia orientalis.
12. The vector of item 11, wherein the RNA polymerase III promoter and/or RNA polymerase III terminator is as defined in any one of items 1 to 5.
13. The vector of any one of items 9 to 12, further comprising: (i) a polynucleotide encoding a protein of interest, operably linked to the RNA polymerase II promoter and the RNA polymerase II terminator; and/or (ii) a polynucleotide encoding an ncRNA, operably linked to the RNA polymerase III promoter and the RNA polymerase III terminator.
14. The vector of item 13, wherein: (i) the protein of interest is or comprises a ribonucleoprotein, an endonuclease, an RNA-guided endonuclease, a CRISPR endonuclease, a type I CRISPR endonuclease, a type II CRISPR endonuclease, a type III CRISPR endonuclease, a type IV CRISPR endonuclease, a type V CRISPR endonuclease, a type VI CRISPR endonuclease, CRISPR associated protein 9 (Cas9), Cpf1, CasX, or CasY; and/or (ii) the ncRNA is or comprises a guideRNA (gRNA), or a crRNA and a tracrRNA.
15. The vector of any one of items 6 to 14, further comprising: (a) a yeast and/or fungal selectable marker; (b) a bacterial selectable marker; (c) a bacterial origin of replication; or (d) any combination of (a)-(c).
16. The vector of item 15, wherein the yeast and/or fungal selectable marker is a positive or negative selectable marker, and/or the bacterial selectable marker is a positive or negative selectable marker.
17. The vector of any one of items 6 to 16, which is a plasmid.
18. The vector of item 17, wherein the plasmid has a size less than 30 kb, 25 kb, 20 kb, 15 kb, 14 kb, 13 kb, 12 kb, 11 kb, 10 kb, 9 kb, 8 kb, 7 kb, 6 kb, or 5 kb.
19. A vector comprising the expression cassette as defined in any one of items 1 to 6.
20. The vector of item 19, which is the vector as defined in any one of items 6 to 10.
21. An expression cassette comprising a polynucleotide encoding a protein of interest, operably linked to the RNA polymerase II promoter as defined in item 10, and/or to the RNA polymerase II terminator as defined in item 10.
22. The expression cassette of item 21, wherein the RNA polymerase II promoter and/or the RNA polymerase II terminator is heterologous to the polynucleotide encoding the protein of interest.
23. A yeast or fungal cell comprising the recombinant DNA molecule as defined in any one of items 1 to 5, the vector as defined in any one of items 6 to 20, or the expression cassette as defined item 21 or 22.
24. Use of the recombinant DNA molecule as defined in any one of items 1 to 5, the vector as defined in any one of items 6 to 20, or the expression cassette as defined item 21 or 22, for genetically engineering host yeast or fungal cells.
25. Use of the recombinant DNA molecule as defined in any one of items 1 to 5, the vector as defined in any one of items 6 to 20, or the expression cassette as defined item 21 or 22, for producing a product of interest from host yeast or fungal cells comprising said recombinant DNA molecule, said vector, or said expression cassette.
26. A method for genetically engineering host yeast or fungal cells, the method comprising transforming the host yeast or fungal cells with the recombinant DNA molecule as defined in any one of items 1 to 5, the vector as defined in any one of items 6 to 20, or the expression cassette as defined item 21 or 22.
27. A method for producing a product of interest from host yeast or fungal cells, the method comprising: (a) providing the yeast or fungal cell as defined in item 23, wherein the yeast or fungal cell produces a product of interest; and (b) culturing said yeast or fungal cell under conditions enabling the synthesis of said product of interest.
28. The use of item 25, or the method of item 27, wherein the product of interest is an organic acid, succinic acid, lactic acid, and/or malic acid.
29. The recombinant DNA molecule of any one of items 1 to 5, the yeast or fungal cell of item 23, the use of item 24, 25 or 28, or the method of item 26, 27 or 28, wherein the host yeast or fungal cell belongs to the species: Issatchenkia orientalis (Pichia kudriavzevii or Candida krusei), Ashbya gossypii, Candida auris, Candida ethanolica, Candida intermedia, Candida orthopsilosis, Candida parapsilosis, Candida sorboxylosa, Candida tanzawaensis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Leptosphaeria biglobosa, Leptosphaeria maculans, Metschnikowia australis, Metschnikowia bicuspidata var. bicuspidata, Millerozyma farinosa, Nakazawaea peltata, Pichia membranifaciens, Pichia pastoris, Pichia sorbitophila, Saccharomycetaceae sp. ‘Ashbya aceri’, Saccharomycopsis fibuligera, Scheffersomyces lignosus, Scheffersomyces shehatae, Scheffersomyces stipitis, Spathaspora girioi, Spathaspora gorwiae, Spathaspora hagerdaliae, Spathaspora passalidarum, Sugiyamaella xylanicola, T. utilis, Tetrapisispora phaffii, Vanderwaltozyma polyspora, or Wickerhamia fluorescens.
30. A method for genetically engineering a yeast or fungal cell, the method comprising: (a) providing a yeast or fungal cell that has been engineered to express a genomically-integrated RNA-guided endonuclease; (b) transforming the yeast or fungal cell with: (i) an expression vector comprising a vector selection marker and a guide RNA (gRNA) operably linked to an RNA polymerase III promoter and terminator, wherein the gRNA is designed to assemble with the RNA-guided endonuclease to cleave at a genomic site of interest; and (ii) a template double-stranded DNA (dsDNA) wherein the template dsDNA is designed to direct repair or edition of the cleaved genomic DNA; and (c) culturing the transformed yeast or fungal cell in selective media and isolating a positive transformant comprising the desired genomic integration of the expression cassette.
31. The method of item 30, further comprising (d) culturing the positive transformant in nonselective media, thereby allowing the positive transformant to lose the expression vector.
32. The method of item 31, further comprising repeating (b) to (d) until the desired level of genetic engineering has been achieved.
33. The method of item 31 or 32, further comprising (e) further transforming the positive transformant with an expression vector and template dsDNA as defined in item 30, which are designed to remove the genomically-integrated RNA-guided endonuclease from the genome of the yeast or fungal cell.
34. The method of item 33, wherein the genomic selection marker is SUC2, LEU2, TRPI, URA3, HIS3, LYS2, or MET15.
35. The method of any one of items 30 to 34, wherein the template dsDNA comprises an expression cassette encoding a protein of interest operably linked to an RNA polymerase II promoter and terminator for expression in the yeast or fungal cell, wherein the template dsDNA is designed to direct repair or edition of the cleaved genomic DNA such that the expression cassette is integrated at the genomic site of interest.
36. The method of any one of items 30 to 35, wherein the expression vector is the vector as defined in any one of items 6 to 20, and/or the yeast or fungal cell is as defined in item 23 or 29.

Other objects, advantages and features of the present description will become more apparent upon the reading of the following non-restrictive description of specific embodiments thereof, given by way of example only with reference to the accompanying drawings.

### EXAMPLES

### Example 1

### Identification of I. orientalis Genomic DNA Fragments Having Autonomously Replicating Activity

An autonomously replicating sequence (ARS) is a relatively small untranscribed DNA sequence that acts as a site for DNA replication. ARSs enable the stable maintenance and inheritance of extrachromosomal DNA, such as a plasmid. In this example, ARSs were identified by first digesting I. orientalis genomic DNA with the restriction enzyme EcoRI, and then cloning the digested genomic DNA (gDNA) fragments into a base plasmid containing a dominant selectable carbon source utilization marker ScSUC2 (invertase gene of Saccharomyces cerevisiae), which enables growth using sucrose as a sole carbon source. Enough gDNA fragment-containing plasmids (clones) were generated to produce a plasmid library that is predicted to cover the I. orientalis genome (about 10 Mb) in duplicate, so as to capture putative ARS-containing gDNA.

The plasmid library containing gDNA fragments was transformed into I. orientalis cells and plated on selective medium (containing sucrose). Plasmids were extracted from successful I. orientalis transformants and re-transformed in cells from at least three different I. orientalis strains to confirm their species-wide functionality. The gDNA-fragments of confirmed plasmids were DNA sequenced.

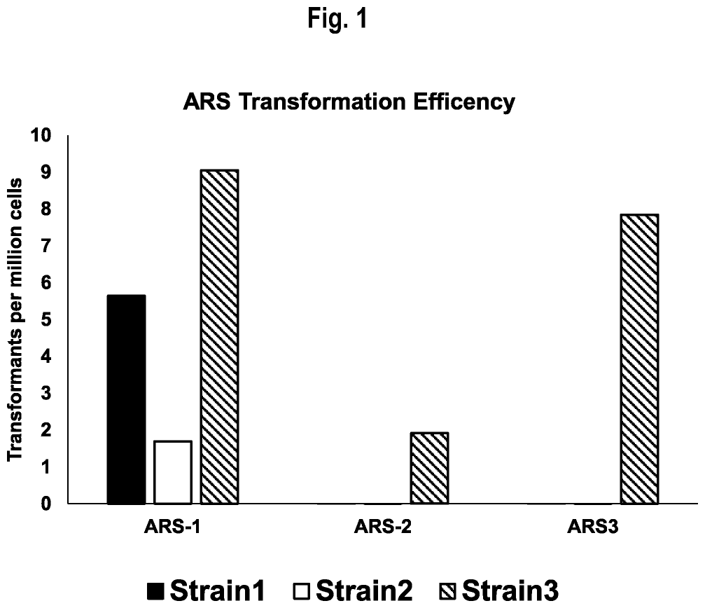

FIG. 1 shows the transformation efficiencies of three plasmids, each having unique ARS-containing gDNA sequences (ARS-1, ARS-2, and ARS-3; SEQ ID NOs: 1, 2, and 3, respectively), which were transformed into three genetically unmodified, wild and distinct I. orientalis isolates (strains 1, 2 and 3), each isolate originating from a different geographic continent.

### Example 2

### Identification of I. orientalis Autonomously Replicating Sequences (ARSs)

### 2.1 ARS-1

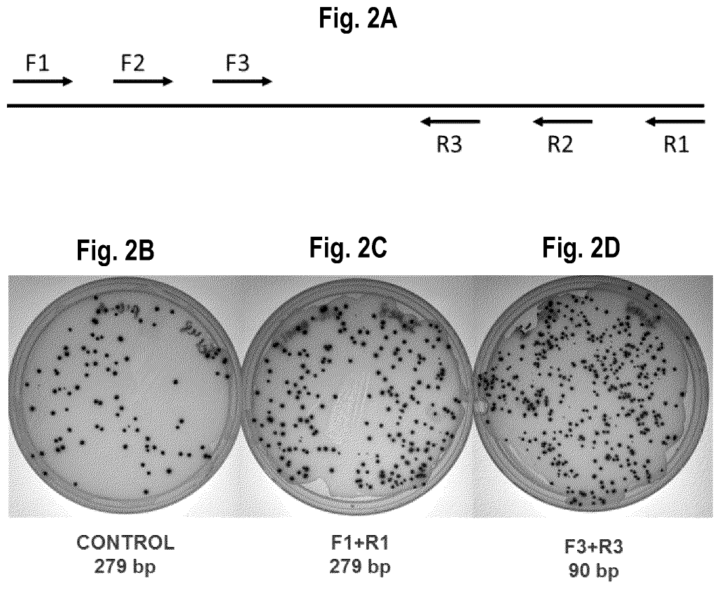

One ARS (ARS-1) resulted in the most efficient transformation efficiency (FIG. 1) and this ARS-containing gDNA fragment was further characterized to identify subregions sufficient to confer autonomously replicating activity. This was performed by PCR amplification of overlapping subregions of the cloned ARS-1-containing DNA (279 bp; black line in FIG. 2A) using different combinations of three forward and reverse primer pairs (arrows in FIG. 2A). PCR amplicons generated from the nine PCR reactions were cloned into the ScSUC2-containing plasmid and transformed into I. orientalis cells. Transformed cells were then plated on sucrose-containing medium and scored for the presence of colony forming units (CFUs) after 48 hours. Plasmids cloned with the smallest amplicon (90 bp), which was generated using Primers F3+R3 (FIG. 2D), were sufficient for successful transformation, and even resulted in higher transformation efficiency than control plasmids cloned with the 279-bp gDNA fragment (FIG. 2B) or with the 279-bp amplicon generated by using Primers F1+R1 (FIG. 2C). The sequence of the 90-bp amplicon sufficient to confer autonomously replicating activity was:

SEQ ID NO: 4
CGAACCCGCAGCCTTTTGATTGCACTTCCTTAACAGAAGAAATCTTA
AGAGTCAAACGCTCTACCGATTGAGCTAACCAGGCTTTTCTTG

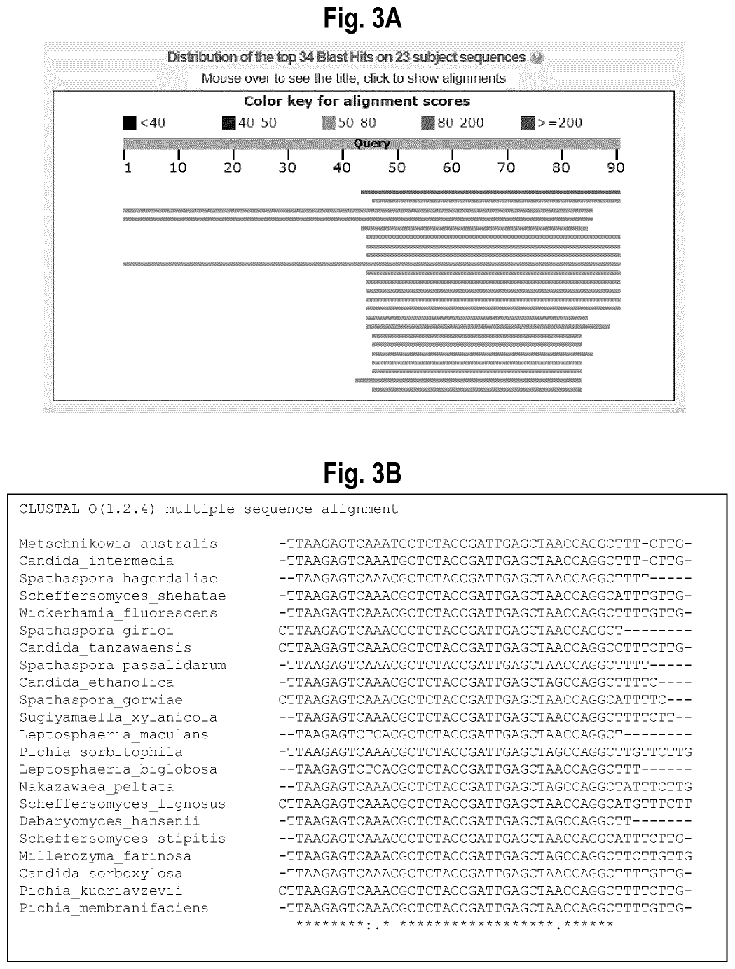

The sequence of the above 90-bp amplicon was analyzed using nucleotide BLAST (nucleotide collection nr/nt): (https://blast.ncbi.nlm.nih.cov/Blast.cgi?PROGRAM=blastn&PAGE_TYPE=BlastSearch&LINK_LOC=blasthome). As shown in FIG. 3A, the analysis revealed that a subregion (around nucleotide positions 46-90) of the 90-bp amplicon sufficient to confer autonomously replicating activity is highly conserved across multiple yeast species. The sequence corresponding to this 45-bp subregion from I. orientalis is:

SEQ ID NO: 5
TAAGAGTCAAACGCTCTACCGATTGAGCTAACCAGGCTTTTCTTG

The above 45-bp subregion was then used as a query sequence in a further nucleotide BLAST analysis (nucleotide collection nr/nt). Analysis and alignment of 1090 blastn hits from 145 unique species further revealed the following consensus sequences:

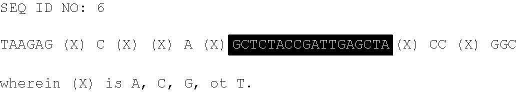

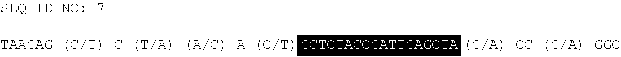

With regard to the above, the core area highlighted in M (SEQ ID NO: 8) comprises positions where sequence identity is greater than 99% across all the 1090 blastn hits analyzed. Consensus nucleotides were generally assigned to a sole nucleotide (i.e., A, C, G, or T) when it was found in at least 80% of the 1090 sequences analyzed. In other cases (where no single consensus nucleotide was assigned), the top two most frequent nucleotides were chosen and the positions are shown in parentheses above for SEQ ID NO: 7.

Table 1 lists examples of different yeast species having significant BLAST alignment scores to the 45-bp query sequence, some of which may have potential industrial applications. A corresponding multiple sequence alignment and phytogenic tree is shown in FIG. 3B and FIG. 3C, respectively.

TABLE 1
List of species with significant BLAST alignment scores
to the 45-bp conserved subregion.

| Species | SEQ ID NO: |
|---|---|
| Candida ethanolica | 9 |
| Candida intermedia | 10 |
| Candida sorboxylosa | 11 |
| Candida tanzawaensis | 12 |
| Debaryomyces hansenii | 13 |
| Leptosphaeria biglobosa | 14 |
| Leptosphaeria maculans | 15 |
| Metschnikowia australis | 16 |
| Millerozyma farinosa | 17 |
| Nakazawaea peltata | 18 |
| Pichia kudriavzevii | 19 |
| Pichia membranifaciens | 20 |
| Pichia sorbitophila | 21 |
| Scheffersomyces lignosus | 22 |
| Scheffersomyces shehatae | 23 |
| Scheffersomyces stipitis | 24 |
| Spathaspora girioi | 25 |
| Spathaspora gorwiae | 26 |
| Spathaspora hagerdaliae | 27 |
| Spathaspora passalidarum | 28 |
| Sugiyamaella xylanicola | 29 |
| Wickerhamia fluorescens | 30 |

Consensus sequences resulting from the multiple sequence alignment shown in FIG. 3B are shown below:

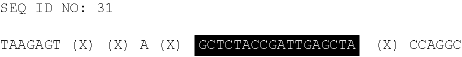

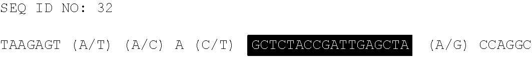

### 2.2 ARS-2

An analogous approach to Example 2.1 can be employed with respect to the gDNA fragment ARS-2 to identify subregions sufficient to confer autonomously replicating activity. Briefly, PCR amplification can be performed of overlapping subregions of the cloned ARS-2-containing DNA using different combinations of forward and reverse primer pairs. The PCR amplicons generated can then be cloned into a ScSUC2-containing plasmid and transformed into I. orientalis cells. Transformed cells can be plated on sucrose-containing medium and scored for the presence of CFUs after 48 hours. Plasmids cloned with the smallest amplicon(s) sufficient for successful transformation (and thus sufficient to confer autonomously replicating activity) can then be sequenced and subjected to nucleotide BLAST analyses to identify regions that are highly conserved across multiple yeast species.

Since a nucleotide BLAST analysis of a 90-bp amplicon of ARS-1 sufficient to confer autonomously replicating activity revealed a highly conserved subregion (see Example 2.1), a similar BLAST analysis was performed for the gDNA fragment ARS-2 (SEQ ID NO: 2). Such an analysis revealed a 73-bp consensus sequence of ARS-2 shown as SEQ ID NO: 70, which was highly conserved (over 85% sequence identity) across multiple species, including the species Ashbya gossypii, Candida auris, Candida intermedia, Candida orthopsilosis, Candida parapsilosis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Metschnikowia bicuspidata var. bicuspidata, Millerozyma farinosa, Pichia kudriavzevii (I. orientalis), Pichia pastoris, Pichia sorbitophila, Saccharomycetaceae sp. ‘Ashbya aceri’, Saccharomycopsis fibuligera, Scheffersomyces stipitis, T. utilis, Tetrapisispora phaffii, and Vanderwaltozyma polyspora (see FIG. 5). More specifically, the sequence set forth as SEQ ID NO: 71 corresponds to a consensus sequence found in 17 different genomic DNA database entries from Pichia kudriavzevii (I. orientalis), including different entries on each of Pichia kudriavzevii chromosomes 1-8 (see FIG. 5). Interestingly, SEQ ID NOs: 70 and 71 were found to contain a 17-bp fragment set forth as SEQ ID NO: 72, which was 100% conserved in all the foregoing species as well as a plurality of other fungal species.

### Example 3

### Identification of Promoters and Terminators of RNA Polymerase II in I. orientalis

The following RNA polymerase II promoters and terminators were identified, cloned and validated in I. orientalis.

TABLE 2
RNA polymerase II promoters and terminators

| I. orientalis sequence | SEQ ID NO: |
|---|---|
| TEF1 Promoter | 33 |
| TDH3 Promoter | 34 |
| PGK1 Promoter | 35 |
| PGI1 Promoter | 36 |
| PFK1 Promoter | 37 |
| PDC1 Promoter | 38 |
| HHF1 Promoter | 39 |
| ENO1 Promoter | 40 |
| CCW12 Promoter | 41 |
| ACT1 Promoter | 42 |
| ADH1 Terminator | 43 |
| TDH3 Terminator | 44 |

### Example 4

### Identification of Promoters of RNA Polymerase III in I. orientalis

Non-polypeptide-coding RNA (ncRNA) can be transcribed into functional RNA molecules in vivo using RNA polymerase III. Transfer RNA (tRNA) sequences function as RNA polymerase III promoters, with transcriptional control sequences (e.g., box A and box B sequences) being intragenic. The I. orientalis tRNA sequences shown in Table 3 were identified based on the analyses of I. orientalis genomic DNA sequences using a publicly available Web tool (http://lowelab.ucsc.edultRNAscan-SE/; Lowe and Chan, 2016; Low and Eddy, 1997), along with other bioinformatic approaches and manual curation.

TABLE 3
I. orientalis RNA polymerase III promoters

| SEQ | | |
|---|---|---|
| ID | | |
| NO: | tRNA | Sequence |
| 45 | Threonine | GCTCGTATGGCCAAGTTGGTAAGGCGCTA |
| | | CACTAGTAATGTAGCGATCCTCAGTTCGA |
| | | CTCTGAGTGCGAGCA |
| 46 | Leucine | GGAGGGATGGCCGAGTGGTCTAAGGCGGC |
| | | AGACTTAAGATCTGTTGGACGCATGTCCG |
| | | CGCGAGTTCGAACCTCGCTTCCTTCA |
| 47 | Proline | GGGTTAATGGTCTAGTGGTATGATTCTCG |
| | | CTTTGGGTGCGAGAGGCCCTGGGTTCAAT |
| | | TCCCAGTTGACCCC |
| 48 | Methionine | GCTTTGGTGGCCCAGTTGGTTAAGGCGTC |
| | | AGTCTCATAATCTGAAGATCGCGAGTTCG |
| | | AATCTCGCCTAGAGCA |
| 49 | Glutamine | TCCGATATAGTGTAACGGCTATCACGGTC |
| | | CGCTTTCACCGGGCAGACCCGGGTTCGAC |
| | | TCCCGGTATCGGAA |
| 50 | Glutamate | AGGTCGTACCCGGATTCGAACCGGGGTTG |
| | | GTCGGATCAAAACCGACAGTGATAACCAC |
| | | TACACTATACAACC |
| 51 | Valine | GGTCGGATGGTCTAGTTGGTTATGGCATA |
| | | TGCTTAACACGCATAACGTCCCCAGTTCG |
| | | ATCCTGGGTTCGATCA |
| 52 | Serine | GGCAATTTGTCCGAGTGGTTAAGGAGAAA |
| | | GATTAGAAATCTTTTGGGCTTTGCCCGCG |
| | | CAGGTTCGAATCCTGCAGTTGTCG |
| 53 | Histidine | GCCGTTCTAGTATAGTGGTCAGTACGCAT |
| | | CGTTGTGGCCGATGAGACCCAGGTTCGAT |
| | | TCCTGGGAACGGCA |
| 54 | Phenylalanine | GCGGGCTTAGCTCAGTGGGAGAGCGCCAG |
| | | ACTGAAGATCTGGAGGCCCTGTGTTCGAT |
| | | CCACAGAGCTCGCA |
| 55 | Arginine | GCCCGTGTAGCGTAATGGTTAACGCGTTT |
| | | GACTTCTAATCAAAAGATTCTGGGTTCGA |
| | | CTCCCAGCATGGGTG |
| 56 | Alanine | GGGCGTGTGGCGTAGTTGGTAGCGCGTTC |
| | | GCCTTGCAAGCGAAAGGTCATCGGTTCGA |
| | | CTCCGGTCTCGTCCA |
| 57 | Isoleucine | GGTCCCTTGGCCCAGTTGGTTAAGGCGTG |
| | | GTGCTAATAACGCCAAGATCAGCAGTTCG |
| | | ATCCTGCTAGGGACCA |
| 58 | Asparagine | CTCCGAGACCGGGAATTGAACCCGGGTCT |
| | | CCCGCGTGACAAGCGGAAATTCTAGCCAC |
| | | TAAACTATCTCGGA |
| 59 | Cysteine | AGCCCGCGGCCGGGTTTGAACCGGCGACC |
| | | AACAGATTTGCAATCTGCTGCTCTACCAC |
| | | TGAGCTACGCGTGC |
| 60 | Tryptophan | GGGGCTATGGCTCAATGGTAGAGCTTTCG |
| | | ACTCCAGATCGAAGGGTTGCAGGTTCGAT |
| | | TCCTGTTGGCCTCA |

Genomic DNA fragments containing tRNA sequences for Threonine, Leucine, and Proline (SEQ ID NOs: 45-47, respectfully) were cloned. In each case, an extra ˜100 bp upstream (5′) of the putative tRNA sequence was included, which facilitated cloning and enabled capture any potential cis-acting 5′ transcription motifs (e.g., TATA box). The cloned sequences including the extra ˜100 bp upstream sequences are shown in SEQ ID NOs: 61-63 for Threonine, Leucine, and Proline, respectively.

### Example 5

### Heterologous Expression of Non-Coding RNA Using RNA Polymerase III Promoters from I. orientalis

Interestingly, attempts at using S. cerevisiae tRNA sequences, such as S. cerevisiae tRNA Tyrosine (SEQ ID NO: 64) and S. cerevisiae tRNA Phenylalanine (SEQ ID NO: 65) failed at expressing non-coding RNA in I. orientalis (negative data not shown). This result was consistent with other observations that standard molecular cloning tools and control sequences that function in traditional yeasts such as S. cerevisiae may not be operable in non-traditional species such as I. orientalis, which are generally regarded as being more difficult to work with.

Accordingly, the ability of several of the tRNA sequences identified in Example 4 to function as RNA polymerase III promoters in I. orientalis was verified herein by evaluating their ability to express a non-coding RNA of interest—i.e., a non-coding guide RNA (gRNA) designed to delete endogenous I. orientalis pyruvate decarboxylase isozyme 1 (IoPDC1) and replace it with a gene encoding the marker GFP. The presence of the pdclA::GFP mutation was used to determine the functionality of the I. orientalis tRNA sequences as RNA polymerase III promoters.

Briefly, the gRNA was cloned into a plasmid containing the I. orientalis ARS of SEQ ID NO: 4 by ligating a 217-bp gRNA expression cassette containing two unique restriction sites. The plasmid containing the gRNA cassette was then transformed into I. orientalis cells that contain a genome-integrated Cas9 expression cassette. Transformants were recovered on plasmid-selective medium. The expressed genome-integrated Cas9 enzyme, which is targeted using the plasmid-based gRNA, generates double-stranded chromosome breaks. The double-stranded DNA break in the chromosome is repaired by co-transforming with the gRNA plasmid and a synthetic double-stranded DNA molecule, which uses homologous recombination to act as a DNA damage repair template.

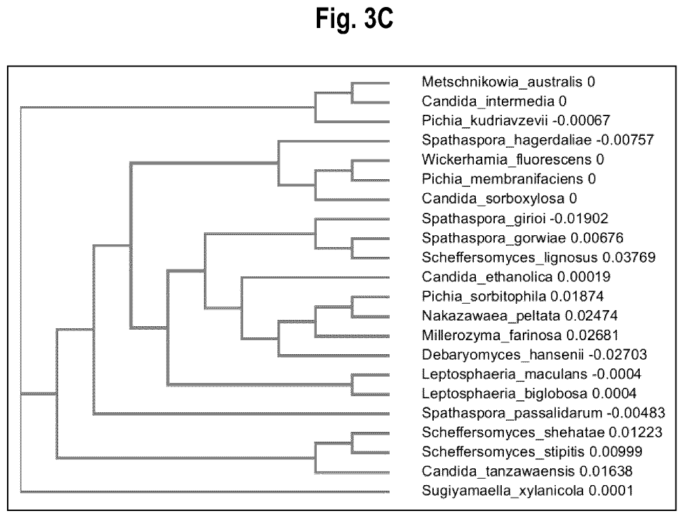

PCR was used to measure the presence of a genome-integrated GFP gene to confirm genome editing. Results are shown in FIG. 4A, FIG. 4B, and FIG. 4C for the tRNA sequences of Threonine, Leucine, and Proline cloned as described in Example 4 (SEQ ID NOs: 61-63), wherein the “A” symbol represents a PCR reaction in which an external primer (outside of IoPDC1) is paired with an internal GFP primer (with IoPDC1), and “wt” represents a PCR reaction in which an external primer is paired with an internal IoPDC1 primer. A wild-type strain containing IoPDC1+ wild-type control is on the far right (“wt control”) of FIG. 4C. The correct integration of the GFP cassette was 100% for each tRNA sequence used (FIG. 4A, FIG. 4B, and FIG. 4C), confirming that the I. orientalis tRNA sequences may be successfully used to express a non-coding RNA of interest.

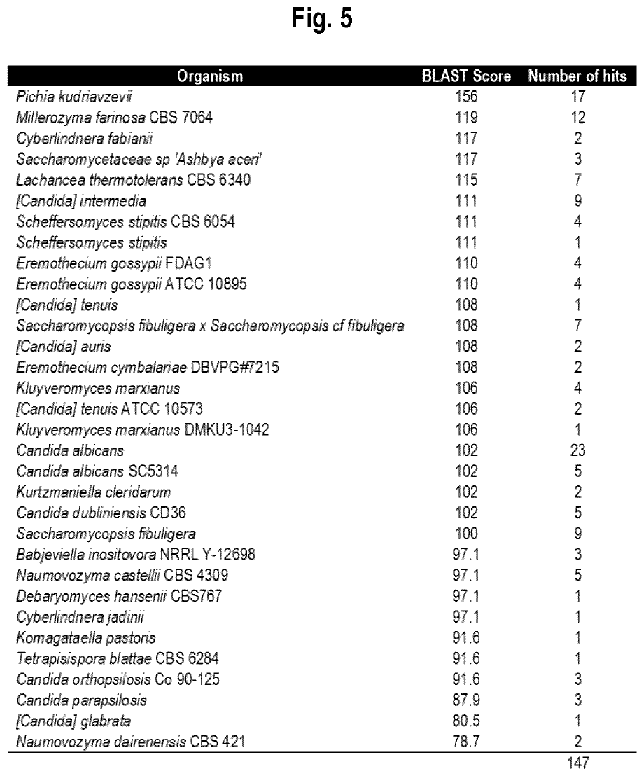

A multiple sequence alignment of the validated I. orientalis tRNA sequences of SEQ ID NOs: 45-47 (shown in FIG. 6) revealed two highly conserved regions (SEQ ID NOs: 66 and 67), which may function as I. orientalis box A and box B RNA polymerase III transcriptional control sequences.

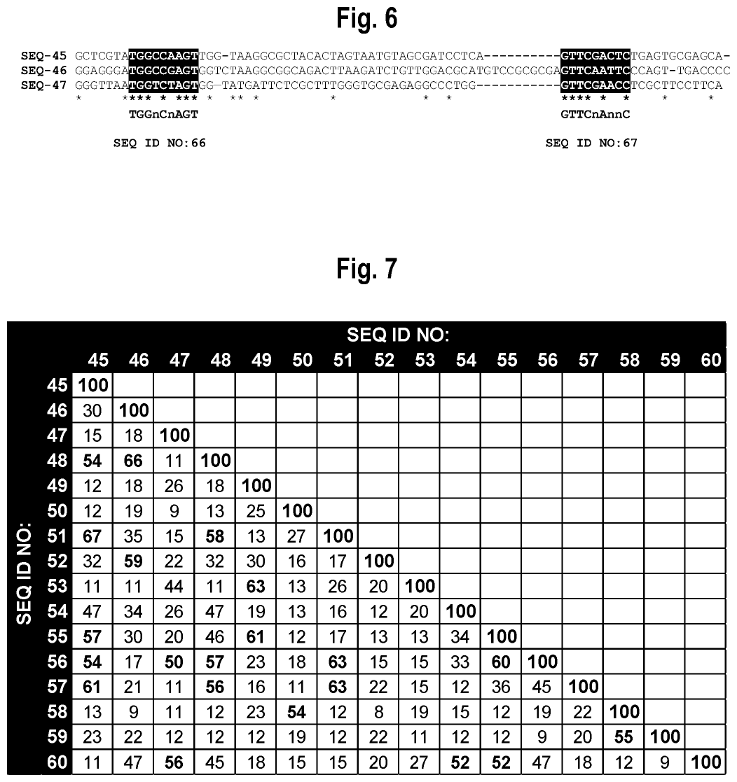

Further multiple sequence alignments of the I. orientalis tRNA sequences listed in Table 3 (SEQ ID NOs: 45-60) revealed structural similarities. Pairwise nucleic acid sequence similarity scores generated using CLUSTALW alignment tool are shown in FIG. 7. Of note, the I. orientalis tRNA threonine sequence (SEQ ID NO: 45) showed alignment scores of at least 54 with each of SEQ ID NOs: 48, 51 and 55-57; the I. orientalis tRNA leucine sequence (SEQ ID NO: 46) showed alignment scores of at least 59 with each of SEQ ID NOs: 48 and 52; and the I. orientalis tRNA proline sequence (SEQ ID NO: 47) showed alignment scores of at least 50 with each of SEQ ID NOs: 56 and 60. Furthermore, all 16 the I. orientalis tRNA sequences listed in Table 3 contained the consensus sequence of GnTCnAnnC (SEQ ID NO: 68), and 15 of the 16 I. orientalis tRNA sequences contained a T at the second position (GTTCnAnnC; SEQ ID NO: 69), which may function as an I. orientalis box B RNA polymerase III transcriptional control sequence.

### Example 6

### Method for Genetically Engineering a Yeast Strain

Transform wild-type I. orientalis with a plasmid containing Cas9 and the gRNA cassette. The gRNA cassette is designed to target URA3 and the repair double-stranded DNA (dsDNA) encodes a Cas9 expression cassette. Homozygous ura3::Cas9/ura3::Cas9 transformants are selected on 5-fluoroorotic acid (5-FOA) medium. Generate a heterozygous, uracil prototrophic strain with the genotype Cas9/URA3 by integrating the URA3 complementation group using standard homologous recombination, and selecting transformants on medium lacking uracil.

This enables genome editing experiments to be performed by the transformation of a plasmid containing only the gRNA (not Cas9), which reduces the plasmid size from >10 kb to approximately 5 kb. Reduced plasmid size vastly increases the transformation and genome editing efficiencies (e.g., 10- to 100-fold) in I. orientalis cells.

Iterative transformation of gRNA-containing plasmid with as dsDNA repair molecule to engineer the genome. Perform four diagnostic PCR confirmations for each gene integration: 1) 5′ confirmation; 2) complete heterologous gene integration; 3) 3′ confirmation; and 4) removal of endogenous wild-type locus.

Transform the Cas9 “suicide guide” containing plasmid. This plasmid targets the genome-integrated Cas9. The cell is restored to URA3/URA3 by homologous recombination by either the homologous chromosome or co-transformed repair dsDNA that encodes the URA3 complementation group (URA3 gene+1000 bp homology).

### REFERENCES

Burstein et al., “New CRISPR-Cas systems from uncultivated microbes”. Nature (2017), 542(7640): 237-241.
Lowe and Eddy, “tRNAscan-SE: A program for improved detection of transfer RNA genes in genomic sequence”. Nucl. Acids Res. (1997), 25: 955-964.
Lowe and Chan, “tRNAscan-SE On-line: Search and Contextual Analysis of Transfer RNA Genes”. Nucl. Acids Res. (2016) 44: W54-57.
Kurtzman et al., The Yeasts: A Taxonomic Study (Fifth Edition), 2010. ISBN: 978-0-444-52149-1
Kurtzman et al., “Emendation of the Genus Issatchenkia kudriavzevii and Comparison of Species by Deoxyribonucleic Aci Reassociation, Mating Reaction and Ascospore Ultrastructure”. International Journal of Systematic Bacteriology, April 1980, p 503-513.
Schramm and Hernandez, “Recruitment of RNA polymerase III to its target promoters.” (2002) Genes Dev. 16:2593-620.

1.-36. (canceled)

37. A vector comprising an autonomously replicating sequence (ARS) having autonomously replicating activity in a yeast or fungal species for an industrial application, the ARS comprising a nucleic acid sequence at least 94% identical to SEQ ID NO: 8.

38. The vector of claim 37, wherein the ARS has at least 99% identity to SEQ ID NO: 8.

39. The vector of claim 37, wherein the ARS further has at least 95% identity to the consensus sequence of SEQ ID NO: 5, 6, 7, 31, or 32.

40. The vector of claim 37, wherein the ARS further has at least 99% identity to the consensus sequence of SEQ ID NO: 5, 6, 7, 31, or 32.

41. The vector of claim 37, wherein the ARS comprises the sequence of SEQ ID NO: 5, 6, 7, 31, or 32.

42. The vector of claim 37, wherein the ARS further has at least 95% identity to SEQ ID NO: 4.

43. The vector of claim 37, wherein the ARS confers autonomously replicating activity to the vector when transformed in a yeast or fungus which is: Issatchenkia orientalis (Pichia kudriavzevii or Candida krusei), Candida ethanolica, Pichia membranifaciens, Candida intermedia, Pichia sorbitophila, Candida sorboxylosa, Scheffersomyces lignosus, Candida tanzawaensis, Scheffersomyces shehatae, Debaryomyces hansenii, Scheffersomyces stipitis, Leptosphaeria biglobosa, Spathaspora girioi, Leptosphaeria maculans, Spathaspora gorwiae, Metschnikowia australis, Spathaspora hagerdaliae, Millerozyma farinosa, Spathaspora passalidarum, Nakazawaea peltata, Sugiyamaella xylanicola, Wickerhamia fluorescens, or any combination thereof.

44. The vector of claim 37, further comprising: (i) a promoter and/or a terminator; (ii) an RNA polymerase II promoter and an RNA polymerase II terminator; (iii) an RNA polymerase III promoter and an RNA polymerase III terminator; or (iv) both (ii) and (iii).

45. The vector of claim 37, further comprising: (a) a yeast and/or fungal selectable marker; (b) a bacterial selectable marker; (c) a bacterial origin of replication; or (d) any combination of (a)-(c).

46. The vector of claim 45, which is a plasmid.

47. A method for genetically engineering host yeast or fungal cells, the method comprising transforming the host yeast or fungal cells with the vector as defined in claim 37, wherein the ARS confers autonomously replicating activity to the vector when transformed in the yeast or fungal cells, and wherein the vector further comprises a polynucleotide to be expressed in the the yeast or fungal cells operably linked to a suitable promoter and terminator.

48. A vector comprising an autonomously replicating sequence (ARS) having autonomously replicating activity in a yeast or fungal species for an industrial application, the ARS comprising a nucleic acid sequence at least 95% identical to SEQ ID NO: 72.

49. The vector of claim 48, wherein the ARS has at least 99% identity to SEQ ID NO: 72.

50. The vector of claim 48, wherein the ARS further has at least 85% identity to the consensus sequence of SEQ ID NO: 70 or 71.

51. The vector of claim 48, wherein the ARS further has at least 90% identity to the consensus sequence of SEQ ID NO: 70 or 71.

52. The vector of claim 48, wherein the ARS further has at least 95% identity to the consensus sequence of SEQ ID NO: 70 or 71.

53. The vector of claim 48, further comprising: (i) a promoter and/or a terminator; (ii) an RNA polymerase II promoter and an RNA polymerase II terminator; (iii) an RNA polymerase III promoter and an RNA polymerase III terminator; or (iv) both (ii) and (iii).

54. The vector of claim 48, further comprising: (a) a yeast and/or fungal selectable marker; (b) a bacterial selectable marker; (c) a bacterial origin of replication; or (d) any combination of (a)-(c).

55. The vector of claim 48, wherein the ARS confers autonomously replicating activity to the vector when transformed in a yeast or fungus which is: Issatchenkia orientalis (Pichia kudriavzevii or Candida krusei), Ashbya gossypii, Candida auris, Candida intermedia, Candida orthopsilosis, Candida parapsilosis, Candida tenuis, Cyberlindnera fabianii, Debaryomyces hansenii, Eremothecium cymbalariae, Kluyveromyces marxianus, Komagataella pastoris, Komagataella phaffii, Lachancea thermotolerans, Metschnikowia bicuspidata var. bicuspidata, Millerozyma farinosa, Pichia pastoris, Pichia sorbitophila, Saccharomycetaceae sp. ‘Ashbya aceri’, Saccharomycopsis fibuligera, Scheffersomyces stipitis, T. utilis, Tetrapisispora phaffli, Vanderwaltozyma polyspora, or any combination thereof.

56. A method for genetically engineering host yeast or fungal cells, the method comprising transforming the host yeast or fungal cells with the vector as defined in claim 48, wherein the ARS confers autonomously replicating activity to the vector when transformed in the yeast or fungal cells, and wherein the vector further comprises a polynucleotide to be expressed in the the yeast or fungal cells operably linked to a suitable promoter and terminator.
